(19) United States
(12) Patent Application Publication
(10) Pub. No.: US 2016/0368895 A1
(43) Pub. Date: Dec. 22, 2016

(54) PULMONARY HYPERTENSION TREATING AGENT

(71) Applicants: THE UNIVERSITY OF TOKYO, Tokyo (JP); NATIONAL UNIVERSITY CORPORATION OKAYAMA UNIVERSITY, Okayama (JP)

(72) Inventors: Tetsuo NAGANO, Tokyo (JP); Takayoshi OKABE, Tokyo (JP); Hirotatsu KOJIMA, Tokyo (JP); Toshifumi SUZUKI, Tokyo (JP); Kenjiro HANAOKA, Tokyo (JP); Manabu SHIMONISHI, Tokyo (JP); Shinichiro EGASHIRA, Tokyo (JP); Hirofumi NAKANO, Tokyo (JP); Kazuo KUMAGAI, Tokyo (JP); Hiroyuki MIYACHI, Okayama (JP); Kenji MATSUNO, Okayama (JP); Minoru WAKI, Okayama (JP)

(73) Assignees: THE UNIVERSITY OF TOKYO, Tokyo (JP); NATIONAL UNIVERSITY CORPORATION OKAYAMA UNIVERSITY, Okayama (JP)

(21) Appl. No.: 15/037,152

(22) PCT Filed: Nov. 27, 2014
(86) PCT No.: PCT/JP2014/081314

§ 371 Date: Sep. 7, 2016

(30) Foreign Application Priority Data
Nov. 29, 2013 (JP) 2013-247446

(51) Int. Cl. C07D 401/06; C07D 495/04; C07D 401/14; C07D 409/14; C07D 407/14

(52) U.S. Cl. CPC C07D 401/06; C07D 409/14; C07D 407/14; C07D 401/14; C07D 495/04

(57) ABSTRACT

[Problem] The present invention addresses the problem of providing a compound that is useful for preventing or treating diseases associated with malfunctioning of PGI2 receptors, in particular, pulmonary hypertension.

[Solution] A positive allosteric regulator of a PGI2 receptor which comprises a compound represented by formula (1) or a pharmaceutically acceptable salt, hydrate or solvate thereof. (1) (In the formula, R1 is a branched chain or cyclic alkyl or alkenyl having 3 to 10 carbon atoms which may be substituted, R2 is a hydrogen atom or an alkyl having 1 to 6 carbon atoms which may be substituted, R3 is 1 to 4 substituents which are the same or different independently selected from the group consisting of a hydrogen atom, a halogen atom, an alkyl having 1 to 6 carbon atoms which may be substituted, and an alkoxy having 1 to 6 carbon atoms which may be substituted, and A is an aryl which may be substituted or a heteroaryl which may be substituted.)

### TECHNICAL FIELD

The present invention relates to a compound that is useful for preventing or treating disease relating to functional depression of PGI₂ receptors, i.e., pulmonary hypertension.

### BACKGROUND ART

Pulmonary hypertension (also referred to as “PH” below) is the generic name for diseases in which the blood pressure of the pulmonary artery is increased, and most pulmonary hypertension is secondary to various types of cardiopulmonary disease. Pulmonary hypertension is a “rare disease,” but the vital prognosis from the onset thereof is known to be an average of three years. Although pulmonary hypertension is generally asymptomatic in the early stages of onset thereof, shortness of breath on exertion, difficulty breathing on exertion, and other symptoms appear as the disease progresses. When pulmonary hypertension becomes further advanced, hypertrophy and enlargement of the right ventricle occur, and are complicated by right ventricular failure in worst cases, resulting in death.

Meanwhile, PGI₂ (prostacyclin) is a lipid molecule which elevates the concentration of intracellular cAMP by binding to PGI₂ receptors, which are G-protein-coupled receptors, and is known to have strong vasodilating action and to have pharmacological action such as platelet aggregation inhibition or inhibitory effects on vascular smooth muscle proliferation. Levels of PGI₂ have been confirmed to be low in patients with pulmonary hypertension, and procedures whereby a PGI₂ receptor agonist, i.e., PGI₂ and a derivative thereof, is administered by intravenous injection or other means (see Patent Reference 1, for example) have recently been performed as methods for treating pulmonary hypertension in cases of advanced symptoms. However, existing PGI₂ receptor agonist preparations have a short in-vivo half-life of approximately 5 minutes and are unstable, and when a high dose is administered in order to maintain the blood concentration thereof, the danger arises of side effects such as systemic low blood pressure or hemorrhage. Therefore, current PGI₂ preparations must be administered by continuous intravenous infusion, which creates a significant burden on the patient and leads to extremely poor quality of life (QOL). Consequently, there is a need for an improved administration method or development of a novel therapeutic agent which can be stably be administered orally.

### PRIOR ART REFERENCES

### Patent References

Patent Reference 1: Japanese Laid-Open Patent Application 8-109162

### SUMMARY OF THE INVENTION

### Problems to be Solved by the Invention

The present invention therefore addresses the problem of providing a compound useful for preventing or treating pulmonary hypertension.

### Means Used to Solve the Above-Mentioned Problems

As a result of thoroughgoing investigations aimed at overcoming the abovementioned problems, the inventors newly discovered that a tetrazole derivative binds to a site on the PGI₂ receptor other than where an endogenous ligand binds thereto, and that the tetrazole derivative exhibits “positive allosteric modulator activity” (positive allosteric regulatory activity) for enhancing PGI₂ receptor sensitivity, and perfected the present invention.

Specifically, one aspect of the present invention provides a PGI₂ receptor positive allosteric regulator which comprises a compound selected from any one of Formulas (1) through (3) below or a pharmaceutically acceptable salt, hydrate, or solvate thereof.

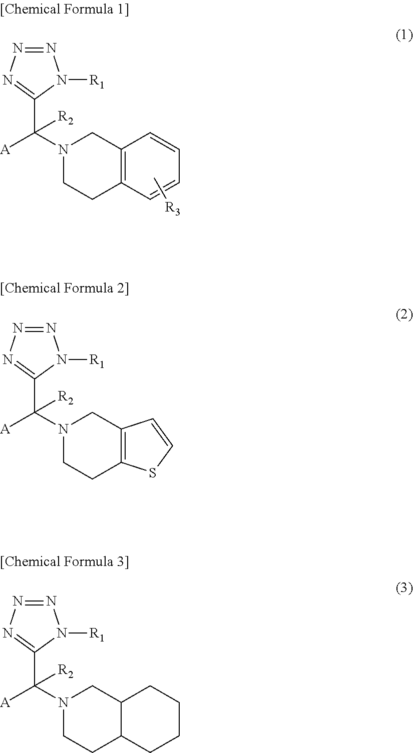

In the above formulas, R¹ represents a branched-chain or cyclic C₃₋₁₀ alkyl or alkenyl which may be substituted, R² represents a hydrogen atom or a C₁₋₆ alkyl which may be substituted, R³ represents 1 to 4 substituents, which are the same or different, independently selected from the group consisting of a hydrogen atom, a halogen atom, a C₁₋₆ alkyl which may be substituted, and a C₁₋₆ alkoxy which may be substituted, and A represents an aryl which may be substituted or a heteroaryl which may be substituted.

In Formulas (1) through (3) above, R¹ is preferably selected from tert-butyl, 1,1-dimethylpropyl, 1-methylcyclopentyl, 1-methylcyclohexyl, 1-methylcycloheptyl, 3-methyl-3-pentyl, and adamantyl. More preferably, R¹ is tert-butyl, 1,1-dimethylpropyl, or 1-methylcyclohexyl.

In the above formulas, R² and R³ are preferably both hydrogen atoms.

In Formulas (1) through (3) above, A is preferably selected from the group consisting of phenyl, thienyl, furyl, pyrrolyl, indolyl, benzothiophenyl, and benzofuranyl, each of which may be substituted.

In a preferred mode, the present invention provides a positive allosteric regulator in which A in Formulas (1) through (3) is a group having the structure indicated below.

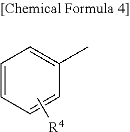

In the formula above, R⁴ represents one to five substituents, which are the same or different, independently selected from the group consisting of a hydrogen atom, a C₁₋₆ alkyl which may be substituted, a C₁₋₆ alkoxy which may be substituted, a halogen atom, a hydroxyl group, a C₁₋₆ alkylthio which may be substituted, an amino which may be substituted, an acetylamino which may be substituted, a silylalkynyl which may be substituted, a benzyloxy which may be substituted, and a nitro, or, when two R⁴ groups are present, the two R⁴ groups may form a saturated or unsaturated ring structure which may include a hetero atom together with carbon atoms to which the R⁴ groups are bonded.

In Formula (2) above, R⁴ is preferably selected from the group consisting of a hydrogen atom, a hydroxy group, an alkoxy group, a benzyloxy group, and an alkylthio group. More preferably, at least one R⁴ is a hydrogen atom and at least one other R⁴ is a 2-hydroxy group, a 2-alkoxy group, a 2-benzyloxy group, or a 2-alkylthio group.

One aspect of the present invention also provides a novel tetrazole derivative or a pharmaceutically acceptable salt, hydrate, or solvate thereof which exhibits positive allosteric modulator activity on PGI₂ receptors.

Another aspect of the present invention provides a medical composition for treating or preventing disease induced by functional depression of PGI₂ receptors, the medical composition comprising the positive allosteric regulator described above. The disease is preferably pulmonary hypertension. The present invention also provides a platelet aggregation inhibitor comprising the positive allosteric regulator described above.

The present invention also provides a combination medicine for treating or preventing disease induced by functional depression of PGI₂ receptors, the combination medicine comprising (A) the positive allosteric regulator described above and (B) a PGI₂ receptor agonist. Preferably, the PGI₂ receptor agonist is selected from the group consisting of epoprostenol, iloprost, cicaprost, beraprost, ibudilast, ozagrel, isbogrel, carbaprostacyclin, clinprost, ataprost, ciprostene, naxaprostene, taprostene, pimilprost, and phthalazinol. The disease is preferably pulmonary hypertension.

Another aspect of the present invention provides the use of a compound represented by Formulas (1) through (3) above to manufacture a medicine for treating or preventing disease induced by functional depression of PGI₂ receptors.

### Effects of the Invention

The present invention can provide a novel medicine for preventing or treating pulmonary hypertension or another disease originating from functional depression of PGI₂ receptors, the medicine having as an active ingredient a tetrazole derivative having positive allosteric modulator activity on PGI₂ receptors. By utilizing such allosteric action, a parent nucleus different from that of the conventional PGI₂ agonist can be acquired, an increased possibility of oral administration can be anticipated, and blood pressure control suitable for in-vivo regulation is made possible. Because an unstable PGI₂ preparation is not administered, or because the administered dose of the PGI₂ preparation can be significantly reduced by using the present invention jointly with the PGI₂ preparation, it is possible to avoid unwanted side effects, improve pathology, and achieve a reduction of patient burden in the administration procedure. More specifically, the tetrazole derivative discovered in the present invention binds to PGI₂ receptors and acts at μM levels, and has the excellent effect of enhancing sensitivity of PGI₂ receptors to an agonist by a factor of 10 or greater.

### BRIEF DESCRIPTION OF THE DRAWINGS

FIG. 1 Graph showing the epoprostenol-cAMP production curve for the presence of various concentrations of the tetrazole derivative.

FIG. 2 Graph showing vasoconstriction inhibition in the presence and absence of the tetrazole derivative of the present invention.

### EMBODIMENTS FOR CARRYING OUT THE INVENTION

Embodiments of the present invention are described below. The embodiments described below do not restrict the scope of the present invention, and changes other than according to the cited examples below may also be implemented as appropriate in a range not compromising the intent of the present invention.

### 1. DEFINITIONS

In the present specification, “positive allosteric regulation” or “positive allosteric modulator activity” means binding to an allosteric binding site rather than to an orthosteric binding site of a PGI₂ receptor and strengthening the in-vivo response to a PGI₂ receptor orthosteric regulatory substance (PGI₂ receptor agonist). Here, an “orthosteric binding site” is a location where an endogenous ligand (agonist) of the PGI₂ receptor binds, and an “allosteric binding site” is a location on the receptor other than where the endogenous ligand binds, and is a location at which binding of a substance causes receptor functioning to be affected. The term “positive allosteric regulator” or “positive allosteric modulator” refers to a compound or a composition comprising the compound which exhibits the abovementioned “positive allosteric regulation” or “positive allosteric modulator activity.”

In the present specification, the term “halogen atom” means a fluorine atom, a chlorine atom, a bromine atom, or an iodine atom.

In the present specification, an “alkyl” may be any aliphatic hydrocarbon group comprising a straight chain, a branched chain, a ring, or any combination thereof. The carbon number of the alkyl group is not particularly limited, and may be, e.g., 1 to 20 (C₁₋₂₀). When a carbon number is specified, what is meant is an “alkyl” having a number of carbons in the specified numerical range. For example, C₁₋₈ alkyl includes methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. In the present specification, alkyl groups may have one or more of any substituent. Alkoxy groups, halogen atoms, amino groups, mono- or di-substituted amino groups, substituted silyl groups, or acyl groups and the like can be cited as examples of substituents, but the possible substituents are not thus limited. When an alkyl group has two or more substituents, the substituents may be the same or different. The same applies for the alkyl portions of other substituents which include an alkyl portion (e.g., alkoxy groups, arylalkyl groups, and the like).

In the present specification, “alkenyl” refers to a straight-chain or branched-chain hydrocarbon group having at least one carbon-carbon double bond. Examples thereof include vinyl, allyl, 1-propenyl, isopropenyl, 1-butenyl, 2-butenyl, 3-butenyl, 1,3-butanedienyl, 1-pentenyl, 2-pentenyl, 3-pentenyl, 4-pentenyl, 1,3-pentanedienyl, 1-hexenyl, 2-hexenyl, 3-hexenyl, 4-hexenyl, 5-hexenyl, and 1,4-hexanedienyl. The double bond may be in cis conformation or trans conformation.

In the present specification, “aryl” may refer to any monocyclic or condensed polycyclic aromatic hydrocarbon group, and may be an aromatic heterocycle comprising one or more hetero atoms (e.g., oxygen atoms, nitrogen atoms, sulfur atoms, or the like) as annular atoms. In this case, the group is sometimes referred to as a “heteroaryl” group or a “heteroaromatic” group. Bonding may occur at all possible positions whether the aryl has a monocyclic or a condensed ring structure. Non-limiting examples of monocyclic aryls include phenyl, thienyl (2- or 3-thienyl), pyridyl, furyl, thiazolyl, an oxazolyl, a pyrazolyl, 2-pyrazinyl, pyrimidinyl, pyrrolyl, imidazolyl, pyridazinyl, 3-isothiazolyl, 3-isoxazolyl, 1,2,4-oxadiazole-5-yl or 1,2,4-oxadiazole-3-yl, and other groups. Non-limiting examples of condensed polycyclic aryls include 1-naphthyl, 2-naphthyl, 1-indenyl, 2-indenyl, 2,3-dihydroindene-1-yl, 2,3-dihydroindene-2-yl, 2-anthryl, indazolyl, quinolyl, isoquinolyl, 1,2-dihydroisoquinolyl, 1,2,3,4-tetrahydroisoquinolyl, indolyl, isoindolyl, phthalazinyl, quinoxalinyl, benzofuranyl, 2,3-dihydrobenzofuran-1-yl, 2,3-dihydrobenzofuran-2-yl, 2,3-dihydrobenzothiophene-1-yl, 2,3-dihydrobenzothiophene-2-yl, benzothiazolyl, benzimidazolyl, fluorenyl or thioxanthenyl, and other groups. In the present specification, an aryl group may have one or more of any substituent on the ring thereof. Alkoxy groups, halogen atoms, amino groups, mono- or di-substituted amino groups, substituted silyl groups, or acyl groups and the like can be cited as examples of substituents, but the possible substituents are not thus limited. When an aryl group has two or more substituents, the substituents may be the same or different. The same applies for the aryl portions of other substituents which include an aryl portion (e.g., aryloxy groups, arylalkyl groups, and the like).

In the present specification, the term “arylalkyl” signifies an alkyl substituted with an abovementioned aryl. The arylalkyl may have one or more of any substituent. Alkoxy groups, halogen atoms, amino groups, mono- or di-substituted amino groups, substituted silyl groups, or acyl groups and the like can be cited as examples of substituents, but the possible substituents are not thus limited. When an acyl group has two or more substituents, the substituents may be the same or different. Non-limiting examples of arylalkyls include benzyl, 2-thienylmethyl, 3-thienylmethyl, 2-pyridylmethyl, 3-pyridylmethyl, 4-pyridylmethyl, 2-furylmethyl, 3-furylmethyl, 2-thiazolylmethyl, 4-thiazolylmethyl, 5-thiazolylmethyl, 2-oxazolylmethyl, 4-oxazolylmethyl, 5-oxazolylmethyl, 1-pyrazolylmethyl, 3-pyrazolylmethyl, 4-pyrazolylmethyl, 2-pyrazinylmethyl, 2-pyrimidinylmethyl, 4-pyrimidinylmethyl, 5-pyrimidinylmethyl, 1-pyrrolylmethyl, 2-pyrrolylmethyl, 3-pyrrolylmethyl, 1-imidazolylmethyl, 2-imidazolylmethyl, 4-imidazolylmethyl, 3-pyridazinylmethyl, 4-pyridazinylmethyl, 3-isothiazolylmethyl, 3-isoxazolylmethyl, 1,2,4-oxadiazole-5-ylmethyl or 1,2,4-oxadiazole-3-ylmethyl, and other groups.

In the same manner, the term “arylalkenyl” in the present specification signifies an alkenyl substituted with an abovementioned aryl.

In the present specification, an “alkoxy group” is a structure in which the aforementioned alkyl group is bonded to an oxygen atom, and a saturated alkoxy group which is straight-chain, branched, cyclic, or a combination thereof can be cited as an example thereof. Preferred examples thereof include methoxy, ethoxy, n-propoxy, isopropoxy, cyclopropoxy, n-butoxy, isobutoxy, s-butoxy, t-butoxy, cyclobutoxy, cyclopropylmethoxy, n-pentyloxy, cyclopentyloxy, cyclopropylethyloxy, cyclobutylmethyloxy, n-hexyloxy, cyclohexyloxy, cyclopropylpropyloxy, cyclobutylethyloxy or cyclopentylmethyloxy, and other groups.

In the present specification, an “aryloxy group” is a group in which an aforementioned aryl group is bonded via an oxygen atom. Examples of aryloxy groups include phenoxy, 2-thienyloxy, 3-thienyloxy, 2-pyridyloxy, 3-pyridyloxy, 4-pyridyloxy, 2-furyloxy, 3-furyloxy, 2-thiazolyloxy, 4-thiazolyloxy, 5-thiazolyloxy, 2-oxazolyloxy, 4-oxazolyloxy, 5-oxazolyloxy, 1-pyrazolyloxy, 3-pyrazolyloxy, 4-pyrazolyloxy, 2-pyrazinyloxy, 2-pyrimidinyloxy, 4-pyrimidinyloxy, 5-pyrimidinyloxy, 1-pyrrolyloxy, 2-pyrrolyloxy, 3-pyrrolyloxy, 1-imidazolyloxy, 2-imidazolyloxy, 4-imidazolyloxy, 3-pyridazinyloxy, 4-pyridazinyloxy, 3-isothiazolyloxy, 3-isoxazolyloxy, 1,2,4-oxadiazole-5-yloxy or 1,2,4-oxadiazole-3-yloxy, and other groups.

In the present specification, an “alkylene” is a divalent group comprising a straight-chain or branched saturated hydrocarbon, and examples thereof include methylene, 1-methylmethylene, 1,1-dimethylmethylene, ethylene, 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, 2,2-di-n-propyltrimethylene, and the like.

In the present specification, an “alkenylene” is a divalent group comprising a straight-chain or branched unsaturated hydrocarbon having at least one carbon-carbon double bond, and examples thereof include ethenylene, 1-methylethenylene, 1-ethylethenylene, 1,2-dimethylethenylene, 1,2-diethylethenylene, 1-ethyl-2-methylethenylene, propenylene, 1-methyl-2-propenylene, 2-methyl-2-propenylene, 1,1-dimethyl-2-propenylene, 1,2-dimethyl-2-propenylene, 1-ethyl-2-propenylene, 2-ethyl-2-propenylene, 1,1-diethyl-2-propenylene, 1,2-diethyl-2-propenylene, 1-butenylene, 2-butenylene, 1-methyl-2-butenylene, 2-methyl-2-butenylene, 1,1-dimethyl-2-butenylene, 1,2-dimethyl-2-butenylene, and the like.

In the present specification, the terms “arylene” and “arylalkylene” mean divalent groups based on the “aryl” and “arylalkyl” groups, respectively, described above. In the same manner, the terms “oxyalkylene” and “aryleneoxy” mean divalent groups based on the “alkoxy” and “aryloxy” groups, respectively, described above.

In the present specification, the terms “alkylamino” and “arylamino” mean amino groups in which a hydrogen atom of an —NH₂ group is substituted with one or two of the alkyl or aryl groups described above. Examples thereof include methylamino, dimethylamino, ethylamino, diethylamino, ethylmethylamino, benzylamino, and the like. In the same manner, the terms “alkylthio” and “arylthio” mean groups in which the hydrogen atom of an —SH group is substituted with an alkyl or aryl group described above. Examples thereof include methylthio, ethylthio, benzylthio, and the like.

The term “amide” used in the present specification includes both RNR′CO— (alkanocarbonyl- when R is an alkyl) and RCONR′— (alkylcarbonylamino- when R is an alkyl).

The term “ester” used in the present specification includes both ROCO— (alkoxycarbonyl- when R is an alkyl) and RCOO— (alkylcarbonyloxy- when R is an alkyl).

In the present specification, the term “ring structure” means a heterocycle or a carbocyclic group when the structure is formed by a combination of two substituents, and such a group may be saturated, unsaturated, or aromatic. Examples thereof include cycloalkyl, phenyl, naphthyl, morpholinyl, piperidinyl, imidazolyl, pyrrolidinyl, pyridyl, and the like. In the present specification, a substituent may form a ring structure with another substituent, and in such cases where substituents are bonded, a person skilled in the art can understand the specific substitution, e.g., the formation of a bond to hydrogen. Consequently, when specific substituents are described as together forming a ring structure, a person skilled in the art can understand that the ring structure can be formed by a normal chemical reaction and easily be generated. Such ring structures as well as the processes for forming the same are within the cognizance of a person skilled in the art.

### 2. Tetrazole Derivative

The PGI₂ receptor positive allosteric regulator of the present invention is characterized by comprising as an active ingredient a tetrazole derivative represented by formulas (1) through (3).

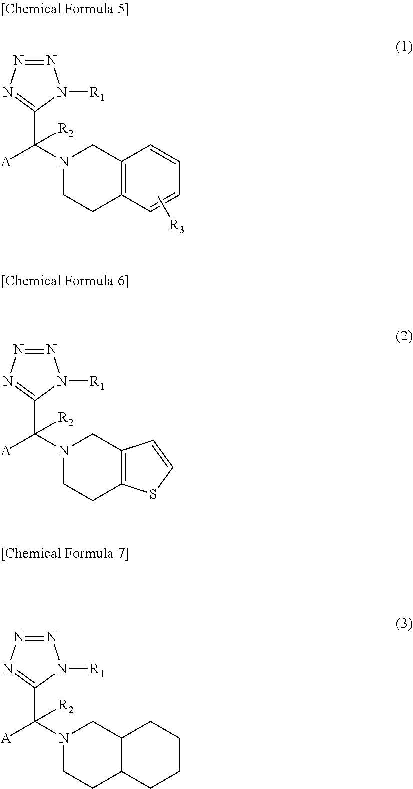

In the above formulas, R¹ represents a branched-chain or cyclic C₃₋₂₀, preferably C₃₋₁₀, alkyl or alkenyl which may be substituted, and the alkyl or alkenyl may be substituted with any substituent. R¹ is preferably a tert-butyl, 1,1-diemthylpropyl, 1-methylcyclopentyl, 1-methylcyclohexyl, 1-methylcycloheptyl, 3-methyl-3-pentyl, or adamantyl group, and is more preferably a tert-butyl, 1,1-dimethylpropyl, or 1-methylcyclohexyl group. R¹ is preferably a bulky functional group.

In the formulas, R² represents a hydrogen atom or a C₁₋₁₀, preferably C₁₋₆, alkyl which may be substituted, and is more preferably a hydrogen atom.

In the formulas, R³ represents 1 to 4 substituents, which are the same or different, independently selected from the group consisting of: a hydrogen atom, a halogen atom, a C₁₋₁₀, preferably C₁₋₆, alkyl which may be substituted; and a C₁₋₁₀, preferably C₁₋₆, alkoxy which may be substituted; and the alkyl and the alkoxy may be substituted with any substituent. Preferably, R³ is a hydrogen atom. Consequently, R² and R³ are both preferably hydrogen atoms.

In the formulas, A represents an aryl which may be substituted or a heteroaryl which may be substituted, and is preferably selected from the group consisting of phenyl, thienyl, furyl, pyrrolyl, indolyl, benzothiophenyl, and benzofuranyl, each of which may be substituted.

More preferably, A in formulas (1) through (3) above is a group having the structure indicated below.

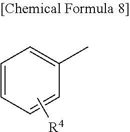

As a specific example, in a mode in which A is a phenyl group in a compound represented by Formula (1), the compound can be represented by Formula (4) below.

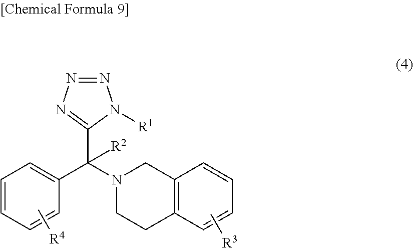

In the formula above, R¹, R², and R³ are the same as defined above.

In the formula above, R⁴ represents one to five, preferably two, same or different substituents independently selected from the group consisting of: a hydrogen atom; a C₁₋₁₀, preferably C₁₋₆, alkyl which may be substituted; a C₁₋₁₀, preferably C₁₋₆, alkoxy which may be substituted; a halogen atom; a hydroxyl group; a C₁₋₁₀, preferably C₁₋₆, alkylthio which may be substituted; an amino which may be substituted; an acetylamino which may be substituted; a silylalkynyl which may be substituted; a benzyloxy which may be substituted; and a nitro. R⁴ is preferably a hydrogen atom, a hydroxy group, a methoxy group, a benzyloxy group, or a methylthio group, and more preferably, at least one R⁴ is a hydrogen atom and at least one other R⁴ is a 2-hydroxy group, a 2-methoxy group, a 2-benzyloxy group, or a 2-methylthio group.

In the formula, R⁴ is preferably in an ortho position with respect to the junction of the phenyl group, and when there are two or more R⁴ groups, at least one R⁴ is preferably in an ortho position. An alkoxy is preferably present in the ortho position.

When two or more R⁴ groups are present, two of the R⁴ groups, preferably two adjacent R⁴ groups, may also form a saturated or unsaturated ring structure which may include a hetero atom together with carbon atoms to which the R⁴ groups are bonded.

The positive allosteric regulator of the present invention includes not only a tetrazole derivative represented by Formulas (1) through (3) above, but also a salt, solvate, or hydrate thereof. The salt is not particularly limited insofar as the salt is a pharmaceutically acceptable salt, and base addition salts, acid addition salts, amino acid salts, and the like can be cited as examples thereof. Sodium salts, potassium salts, calcium salts, magnesium salts, and other alkaline earth metal salts; ammonium salts; or triethylamine salts, piperidine salts, morpholine salts, and other organic amine salts can be cited as examples of base addition salts; and hydrochlorides, hydrobromates, sulfates, nitrates, phosphates, and other mineral acid salts; and salts of methanesulfonic acid, benzenesulfonic acid, p-toluenesulfonic acid, acetic acid, propionic acid, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, salicylic acid, and other organic acids can be cited as examples of acid addition salts. Glycinates, asparaginates, glutamates, and the like can be cited as examples of amino acid salts. The salt may also be an aluminum salt or other metal salt.

The type of solvent for forming a solvate is not particularly limited, but ethanol, acetone, isopropanol, and other solvents can be cited as examples thereof.

Unless otherwise noted, the tetrazole derivatives represented by Formulas (1) through (3) above include tautomers, geometric isomers (e.g., E-isomers, Z-isomers, and the like), enantiomers, and other stereoisomers thereof. Specifically, when one or more asymmetric carbons are included in the tetrazole derivatives represented by Formulas (1) through (3), the stereochemistry of each asymmetric carbon may independently be (R) or (S), and the derivatives may sometimes be present as enantiomers, diastereomers, or other stereoisomers thereof. Consequently, any stereoisomer in pure form, any mixture of stereoisomers, a racemate thereof, or the like can be used as the active ingredient of the positive allosteric regulator of the present invention, and any of these configurations is included in the scope of the present invention.

The compounds below are cited as non-limiting specific examples of tetrazole derivatives represented by Formula (1). 2-((2-(benzyloxy)phenyl)(1-tert-butyl-1H-tetrazol-5-yl)methyl)-1,2,3,4-tetrahydroisoquinoline, 2-((1-tert-butyl-1H-tetrazol-5-yl) (2-methoxyphenyl)methyl)-1,2,3,4-tetrahydroisoquinoline, 2-((1-tert-butyl-1H-tetrazol-5-yl)(4-isopropoxyphenyl)methyl)-1,2,3,4-tetrahydroisoquinoline, 2-((1-tert-butyl-1H-tetrazol-5-yl)(2-chlorophenyl)methyl)-1,2,3,4-tetrahydroisoquinoline, 2-((1-tert-butyl-1H-tetrazol-5-yl)(2-fluorophenyl)methyl)-1,2,3,4-tetrahydroisoquinoline, 2-((1-tert-butyl-1H-tetrazol-5-yl)(2-methylthiophenyl)methyl)-1,2,3,4-tetrahydroisoquinoline, 2-((1-tert-butyl-1H-tetrazol-5-yl)(2-ethoxyphenyl)methyl)-1,2,3,4-tetrahydroisoquinoline, 2-((1-tert-butyl-1H-tetrazol-5-yl)(2-hydroxyphenyl)methyl)-1,2,3,4-tetrahydroisoquinoline, 2-((1-adamantyl-1H-tetrazol-5-yl)(2-methoxyphenyl)methyl)-1,2,3,4-tetrahydroisoquinoline, 2-((2-methoxyphenyl)(1-(1-methylcyclohexyl)-1H-tetrazol-5-yl)methyl)-1,2,3,4-tetrahydroisoquinoline, 2-((1-tert-butyl-1H-tetrazol-5-yl)(thiophen-2-yl)methyl)-1,2,3,4-tetrahydroisoquinoline, 2-((1-tert-butyl-1H-tetrazol-5-yl)(thiophen-3-yl)methyl)-1,2,3,4-tetrahydroisoquinoline, 2-((1-tert-butyl-1H-tetrazol-5-yl)(furan-2-yl)methyl)-1,2,3,4-tetrahydroisoquinoline, 2-((1-tert-butyl-1H-tetrazol-5-yl)(furan-3-yl)methyl)-1,2,3,4-tetrahydroisoquinoline, 2-((1-tert-butyl-1H-tetrazol-5-yl)(1H-indol-3-yl)methyl)-1,2,3,4-tetrahydroisoquinoline,

2-((1-(tert-pentyl)-1H-tetrazol-5-yl)(phenyl)methyl)-1,2,3,4-tetrahydroisoquinoline, and 2-((2-methoxyphenyl)(1-(tert-pentyl)-1H-tetrazol-5-yl)methyl)-1,2,3,4-tetrahydroisoquinoline.

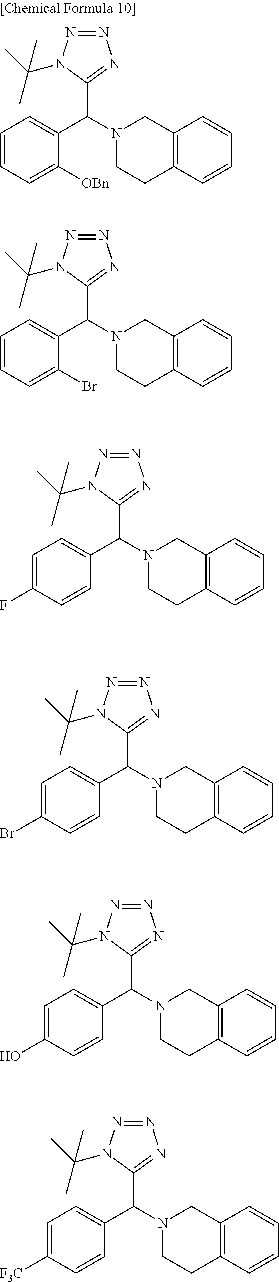 [SMILES: CC(C)(C)n1nnnc1C(c1ccccc1OCc1ccccc1)N1CCc2ccccc2C1 ; CC(C)(C)n1nnnc1C(c1ccccc1Br)N1CCc2ccccc2C1 ; CC(C)(C)n1nnnc1C(c1ccc(F)cc1)N1CCc2ccccc2C1 ; CC(C)(C)n1nnnc1C(c1ccc(Br)cc1)N1CCc2ccccc2C1 ; CC(C)(C)n1nnnc1C(c1ccc(O)cc1)N1CCc2ccccc2C1 ; CC(C)(C)n1nnnc1C(c1ccc(C(F)(F)F)cc1)N1CCc2ccccc2C1]

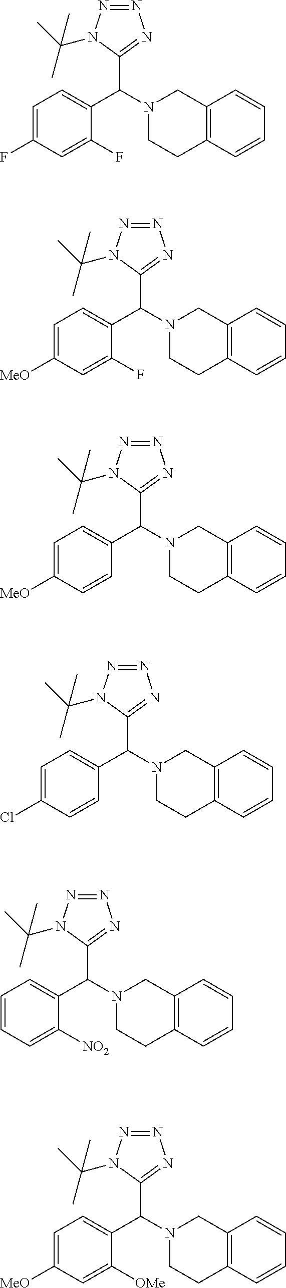 [SMILES: CC(C)(C)n1nnnc1C(c1ccc(F)cc1F)N1CCc2ccccc2C1 ; COc1ccc(C(c2nnnn2C(C)(C)C)N2CCc3ccccc3C2)c(F)c1 ; COc1ccc(C(c2nnnn2C(C)(C)C)N2CCc3ccccc3C2)cc1 ; CC(C)(C)n1nnnc1C(c1ccc(Cl)cc1)N1CCc2ccccc2C1 ; CC(C)(C)n1nnnc1C(c1ccccc1[N+](=O)[O-])N1CCc2ccccc2C1 ; COc1ccc(C(c2nnnn2C(C)(C)C)N2CCc3ccccc3C2)c(OC)c1]

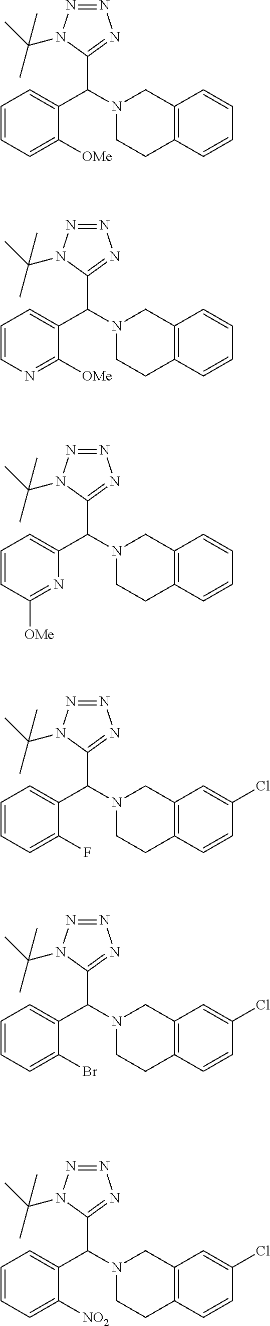 [SMILES: COc1ccccc1C(c1nnnn1C(C)(C)C)N1CCc2ccccc2C1 ; COc1ncccc1C(c1nnnn1C(C)(C)C)N1CCc2ccccc2C1 ; COc1cccc(C(c2nnnn2C(C)(C)C)N2CCc3ccccc3C2)n1 ; CC(C)(C)n1nnnc1C(c1ccccc1F)N1CCc2ccc(Cl)cc2C1 ; CC(C)(C)n1nnnc1C(c1ccccc1Br)N1CCc2ccc(Cl)cc2C1 ; CC(C)(C)n1nnnc1C(c1ccccc1[N+](=O)[O-])N1CCc2ccc(Cl)cc2C1]

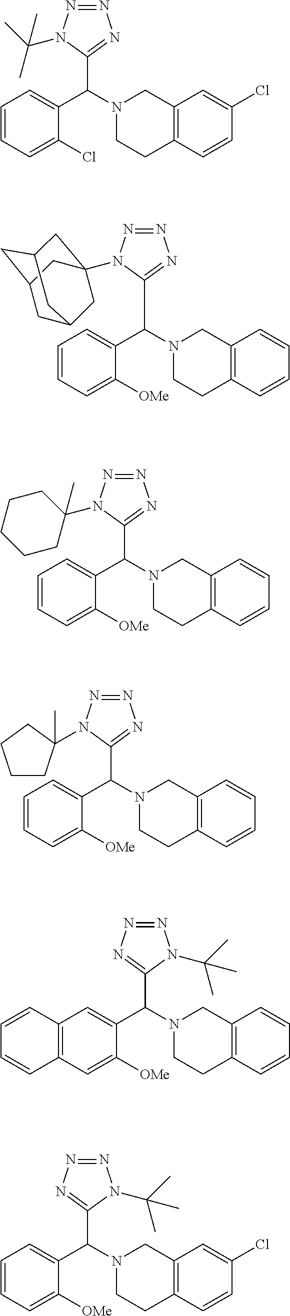 [SMILES: CC(C)(C)n1nnnc1C(c1ccccc1Cl)N1CCc2ccc(Cl)cc2C1 ; COc1ccccc1C(c1nnnn1C12CC3CC(CC(C3)C1)C2)N1CCc2ccccc2C1 ; COc1ccccc1C(c1nnnn1C1(C)CCCCC1)N1CCc2ccccc2C1 ; COc1ccccc1C(c1nnnn1C1(C)CCCC1)N1CCc2ccccc2C1 ; COc1cc2ccccc2cc1C(c1nnnn1C(C)(C)C)N1CCc2ccccc2C1 ; COc1ccccc1C(c1nnnn1C(C)(C)C)N1CCc2ccc(Cl)cc2C1]

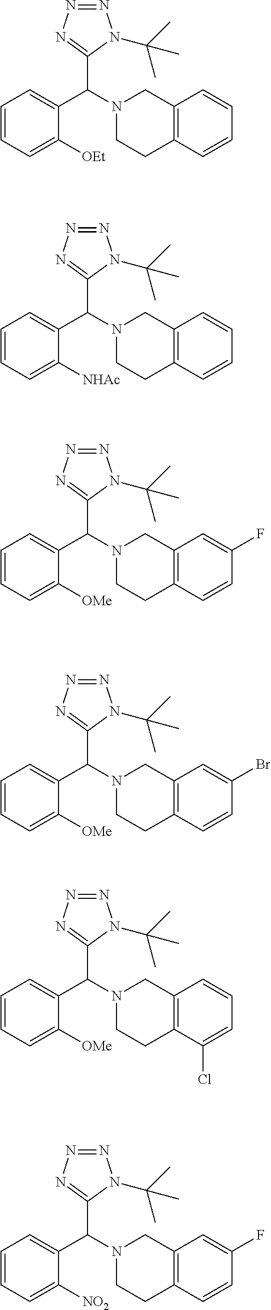 [SMILES: CCOc1ccccc1C(c1nnnn1C(C)(C)C)N1CCc2ccccc2C1 ; CC(=O)Nc1ccccc1C(c1nnnn1C(C)(C)C)N1CCc2ccccc2C1 ; COc1ccccc1C(c1nnnn1C(C)(C)C)N1CCc2ccc(F)cc2C1 ; COc1ccccc1C(c1nnnn1C(C)(C)C)N1CCc2ccc(Br)cc2C1 ; COc1ccccc1C(c1nnnn1C(C)(C)C)N1CCc2c(Cl)cccc2C1 ; CC(C)(C)n1nnnc1C(c1ccccc1[N+](=O)[O-])N1CCc2ccc(F)cc2C1]

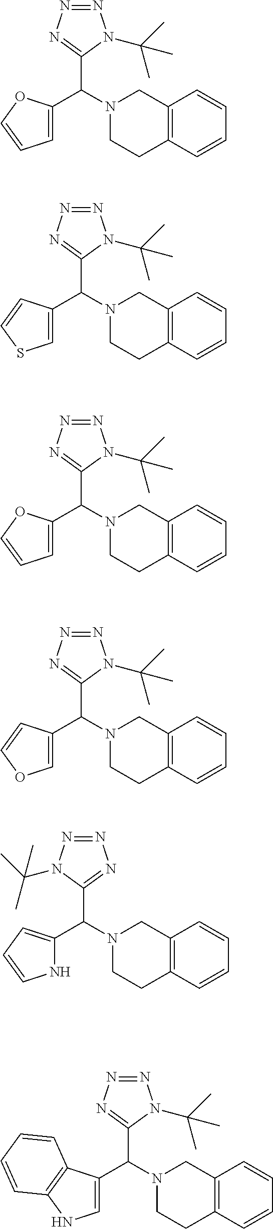 [SMILES: CC(C)(C)n1nnnc1C(c1ccco1)N1CCc2ccccc2C1 ; CC(C)(C)n1nnnc1C(c1ccsc1)N1CCc2ccccc2C1 ; CC(C)(C)n1nnnc1C(c1ccco1)N1CCc2ccccc2C1 ; CC(C)(C)n1nnnc1C(c1ccoc1)N1CCc2ccccc2C1 ; CC(C)(C)n1nnnc1C(c1ccc[nH]1)N1CCc2ccccc2C1 ; CC(C)(C)n1nnnc1C(c1c[nH]c2ccccc12)N1CCc2ccccc2C1]

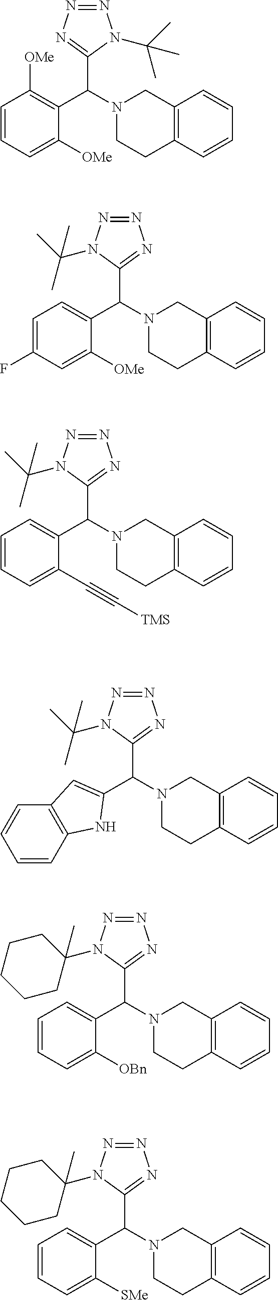 [SMILES: COc1cccc(OC)c1C(c1nnnn1C(C)(C)C)N1CCc2ccccc2C1 ; COc1cc(F)ccc1C(c1nnnn1C(C)(C)C)N1CCc2ccccc2C1 ; CC(C)(C)n1nnnc1C(c1ccccc1C#C[Si](C)(C)C)N1CCc2ccccc2C1 ; CC(C)(C)n1nnnc1C(c1cc2ccccc2[nH]1)N1CCc2ccccc2C1 ; CC1(n2nnnc2C(c2ccccc2OCc2ccccc2)N2CCc3ccccc3C2)CCCCC1 ; CSc1ccccc1C(c1nnnn1C1(C)CCCCC1)N1CCc2ccccc2C1]

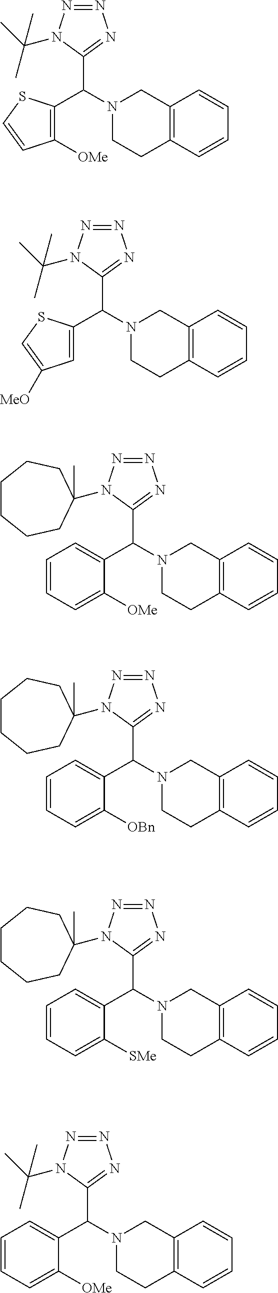 [SMILES: COc1ccsc1C(c1nnnn1C(C)(C)C)N1CCc2ccccc2C1 ; COc1csc(C(c2nnnn2C(C)(C)C)N2CCc3ccccc3C2)c1 ; COc1ccccc1C(c1nnnn1C1(C)CCCCCC1)N1CCc2ccccc2C1 ; CC1(n2nnnc2C(c2ccccc2OCc2ccccc2)N2CCc3ccccc3C2)CCCCCC1 ; CSc1ccccc1C(c1nnnn1C1(C)CCCCCC1)N1CCc2ccccc2C1 ; COc1ccccc1C(c1nnnn1C(C)(C)C)N1CCc2ccccc2C1]

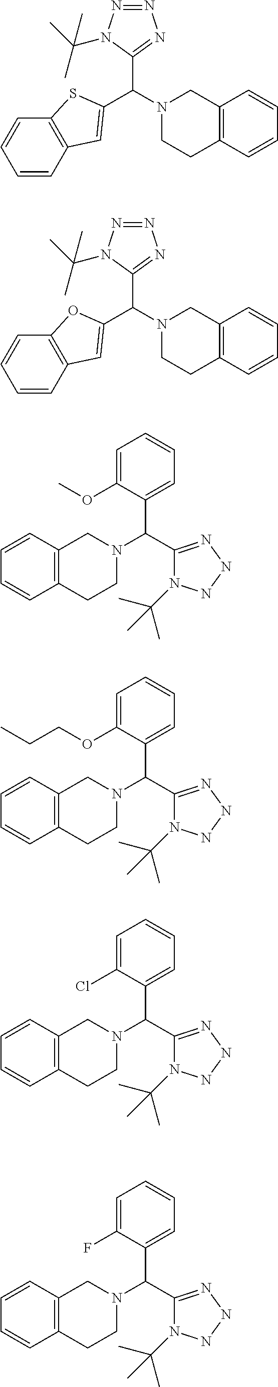

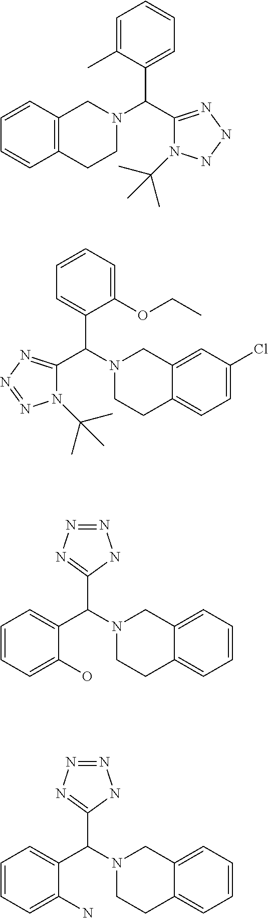 [SMILES: Cc1ccccc1C(C1=NNNN1C(C)(C)C)N1CCc2ccccc2C1 ; CCOc1ccccc1C(c1nnnn1C(C)(C)C)N1CCc2ccc(Cl)cc2C1 ; Oc1ccccc1C(c1nnn[nH]1)N1CCc2ccccc2C1 ; Nc1ccccc1C(c1nnn[nH]1)N1CCc2ccccc2C1]

In addition to a positive allosteric regulator comprising as an active ingredient a tetrazole derivative represented by Formulas (1) through (3) or a pharmaceutically acceptable salt, hydrate, or solvate thereof, the present invention also relates to a medicinal composition and platelet aggregation inhibitor (referred to collectively below as the “medicine of the present invention”) comprising the positive allosteric regulator, for treating or preventing disease induced by functional depression of PGI₂ receptors. The disease induced by functional depression of PGI₂ receptors is preferably pulmonary hypertension.

The medicine of the present invention may be administered as the tetrazole derivative represented by Formulas (1) through (3) or pharmaceutically acceptable salt, hydrate, or solvate thereof itself as the active ingredient, but it is generally preferred that the active ingredient be administered in the form of a medicinal composition comprising the abovementioned substance as the active ingredient and one or more additives for preparation. The term “composition” such as relating the medicinal composition subsumes not only products comprising active components and inactive components (pharmaceutically acceptable excipients) for constituting a carrier, but also any product directly or indirectly generated as a result of association, complexing, or aggregation of any two or more components, as a result of dissociation of one or more components, or as a result of another type of reaction or interaction of one or more components.

A combination of two or more types of tetrazole derivatives represented by Formulas (1) through (3) may be used as the active ingredient of the medicine of the present invention, or another known active ingredient for preventing or treating disease induced by functional depression of PGI₂ receptors may also be blended.

Specifically, the present invention subsumes (A) the abovementioned positive allosteric regulator comprising a tetrazole derivative, as well as (B) a combination medicine for treating or preventing disease induced by functional depression of PGI₂ receptors which includes a PGI₂ receptor agonist. An agonist publicly known in the technical field can be used as such a “PGI₂ receptor agonist,” and epoprostenol, iloprost, cicaprost, beraprost, ibudilast, ozagrel, isbogrel, carbaprostacyclin, clinprost, ataprost, ciprostene, naxaprostene, taprostene, pimilprost, and phthalazinol can be cited as examples thereof.

The type of medicinal composition is not particularly limited, and dosage forms thereof include tablets, capsules, granules, powders, syrups, suspensions, suppositories, ointments, creams, gels, transdermal patches, inhalants, injections, and the like. These preparations are prepared in accordance with the usual methods. A liquid preparation may be dissolved or suspended in water or another appropriate solvent at the time of use. Tablets and granules may be coated by a well-known method. In the case of an injection, the injection is prepared by dissolving the compound of the present invention in water, but the compound of the present invention may also be dissolved in physiological saline or a glucose solution as needed, and a buffer or preservative may also be added. The present invention is provided by any formulation for oral administration or parenteral administration. For example, the present invention can be prepared as a medicinal composition for oral administration in a form such as granules, fine granules, a powder, a hard capsule, a soft capsule, a syrup, an emulsion, a suspension, or a liquid, or as a medicinal composition for parenteral administration in the form of an injection such as for intravenous injection, intramuscular injection, or subcutaneous injection, or in a form such as drops, a percutaneous absorption preparation, a transmucosal absorption preparation, nasal drops, an inhalant, or a suppository. An injection or drops can be prepared as a powdered formulation such as in freeze-dried form and dissolved in physiological saline or another appropriate aqueous medium at the time of use thereof. A sustained-release product coated with a polymer or the like can also be directly administered intracerebrally.

The types of additives for preparation used in manufacturing the medicinal composition, the ratios of additives for preparation with respect to the active ingredient, or the method for manufacturing the medicinal composition can appropriately be selected by a person skilled in the art in accordance with the form of the composition. Inorganic or organic substances, or solid or liquid substances may be used as additives for preparation, and additives for preparation may generally be blended in the range of 1 wt % to 90 wt % with respect to the weight of the active ingredient. Specific examples of such substances include lactose, glucose, mannitol, dextrin, cyclodextrin, starch, sucrose, magnesium aluminometasilicate, synthetic aluminum silicate, sodium carboxymethyl cellulose, hydroxypropyl starch, calcium carboxymethyl cellulose, ion exchange resins, methyl cellulose, gelatin, gum Arabic, hydroxypropyl cellulose, hydroxypropyl methylcellulose, polyvinylpyrrolidone, polyvinyl alcohol, light anhydrous silicic acid, magnesium stearate, talc, tragacanth, bentonite, VEEGUM, titanium oxide, sorbitan fatty acid ester, sodium lauryl sulfate, glycerin, glycerin fatty acid ester, purified lanolin, glycerinated gelatin, polysorbate, macrogol, vegetable oils, waxes, liquid paraffin, white petrolatum, fluorocarbons, non-ionic surfactants, propylene glycol, water, and the like.

To manufacture a solid preparation for oral administration, the active ingredient and an excipient component, e.g., lactose, starch, crystalline cellulose, calcium lactate, silicic anhydride, or the like, are mixed to obtain a powder, or sucrose, hydroxypropyl cellulose, polyvinylpyrrolidone, or another binder and carboxymethyl cellulose, calcium carboxymethyl cellulose, or another disintegrating agent are added as needed and granules are obtained by wet or dry granulation. To manufacture a tablet, the excipients and granules as such may be compressed into tablets, or magnesium stearate, talc, or another lubricant may be added thereto for compression into tablets. The granules or tablets may also be coated with hydroxypropyl methylcellulose phthalate, a methacrylic acid-methyl methacrylate polymer, or another enteric-tablet base and configured as an enteric tablet preparation, or coated with ethyl cellulose, carnauba wax, a hardened oil, or the like and configured as an extended-release preparation. To manufacture a capsule, the excipients or granules may be packed into a hard capsule, or the active ingredient may be coated with a gelatin film without modification or after being dissolved in glycerin, polyethylene glycol, sesame oil, olive oil, or the like to form a soft capsule.

To manufacture an injection, the active ingredient may be dissolved in distilled water for injection together with hydrochloric acid, sodium hydroxide, lactose, lactic acid, sodium, sodium hydrogen phosphate, sodium dihydrogen phosphate, or another pH adjuster, and sodium chloride, glucose, or another isotonizing agent as needed, and sterile-filtered and filled into an ampoule, or mannitol, dextrin, cyclodextrin, gelatin, or the like may be further added and the product vacuum freeze-dried to obtain an injection to be dissolved at the time of use thereof. Lecithin, polysorbate 80, polyoxyethylene hardened castor oil, or the like may also be added to the active ingredient and emulsified in water to obtain an emulsion for injection.

The dosage and number of doses of the medicine of the present invention are not particularly limited, and can appropriately be selected by the judgment of a physician in accordance with the purpose for preventing and/or treating the worsening or progress of the disease to be treated, the type of disease, the body weight or age of the patient, the degree of serious ness of the disease, and other conditions. In general, the orally administered dosage per day for an adult is about 0.01 to 1000 mg (by weight of the active ingredient), and can be administered once a day or divided into multiple doses, or administered once every several days. When an injection is used, continuous or intermittent administration of a daily dose of 0.001 to 100 mg (by weight of the active ingredient) is preferred for an adult.

### 3. Method for Manufacturing the Tetrazole Derivative

The tetrazole derivative of Formula (1) according to the present invention typically can be manufactured by the method described below (Scheme 1).

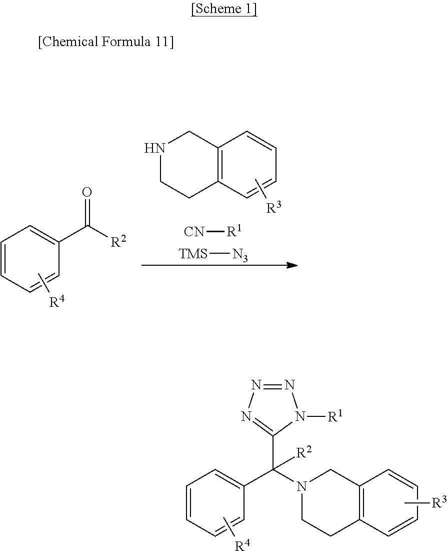

Specifically, the compound of Formula (4) above can be synthesized by reacting, in a solvent, the compound (a) represented by the formula below:

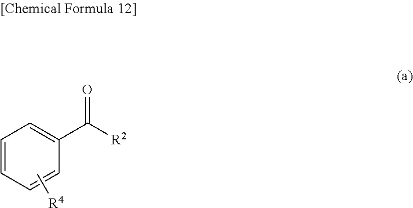

[where R² and R⁴ are the same as defined above], the isocyanide derivative (b) represented by the formula below:

CN—R¹ [Chemical Formula 13]

[where R¹ is the same as defined above], the tetrahydroisoquinoline (c) represented by the formula below:

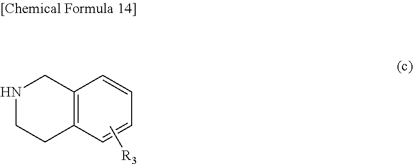

[where R³ is the same as defined above], and trimethylsilyl azide. The tetrazole derivative can be manufactured by the same method also when A in Formula (1) is a group other than a phenyl group. The compounds represented by Formulas (2) and (3) can also be manufactured using the same reaction.

The reaction can be performed in methanol, ethanol, DMF, or another solvent. The reaction can be performed at a temperature of −50° C. to 150° C., and preferably from −20° C. to 100° C. The reaction time is usually 1 to 48 hours, and preferably 4 to 24 hours. However, the solvent, reaction temperature, and other reaction conditions are not limited to the conditions described above, and a person skilled in the art could appropriately select conditions on the basis of knowledge common in the field of organic synthesis.

### EXAMPLES

The present invention will next be described in further detail using examples, but the examples are not limiting of the present invention.

1. Synthesis

The various tetrazole derivatives as the active ingredient of the present invention were synthesized as described below.

### Example 1

### Synthesis of 2-((2-(benzyloxy)phenyl)(1-tert-butyl-1H-tetrazol-5-yl)methyl)-1,2,3,4-tetrahydroisoquinoline

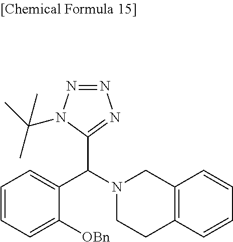 [SMILES: CC(C)(C)n1nnnc1C(c1ccccc1OCc1ccccc1)N1CCc2ccccc2C1]

To a 100-mL recovery flask were added 10 mL of methanol, 0.21 g (1.0 mmol) of 2-benzyloxybenzaldehyde, 0.28 g (2.1 mmol) of 1,2,3,4-tetrahydroisoquinoline, 0.24 g (2.1 mmol) of trimethylsilyl azide, and t-butylisocyanide. The contents of the flask were stirred for 15 hours at room temperature and then concentrated through use of an evaporator. The resultant residue was purified by silica gel column chromatography (n-hexane:AcOEt=5:1 v/v), and 0.45 g (99% yield) of the desired compound was obtained.

### Examples 2 through 56

The compounds shown in Table 1 below were created by the same procedure as in Example 1.

TABLE 1

| Example | Structure | ¹H NMR (300 MHz) | Name |
|---|---|---|---|
| 1 | 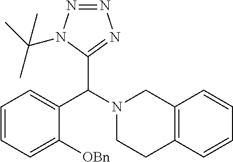 [SMILES: CC(C)(C)n1nnnc1C(c1ccccc1OCc1ccccc1)N1CCc2ccccc2C1] | [symbol] | [symbol] |
| 2 | 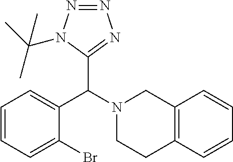 [SMILES: CC(C)(C)n1nnnc1C(c1ccccc1Br)N1CCc2ccccc2C1] | [symbol] | [symbol] |
| 3 | 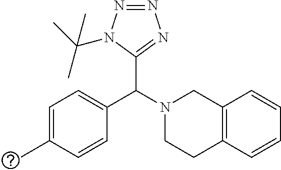 [SMILES: Cc1ccc(C(c2nnnn2C(C)(C)C)N2CCc3ccccc3C2)cc1] | [symbol] | [symbol] |
| 4 | 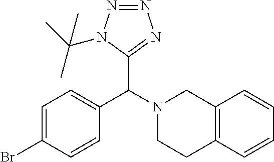 [SMILES: CC(C)(C)n1nnnc1C(c1ccc(Br)cc1)N1CCc2ccccc2C1] | [symbol] | [symbol] |
| 5 | 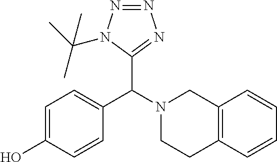 [SMILES: CC(C)(C)n1nnnc1C(c1ccc(O)cc1)N1CCc2ccccc2C1] | [symbol] | [symbol] |
| 6 | 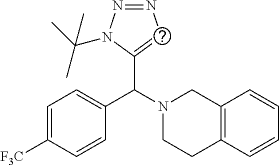 [SMILES: CC(C)(C)n1nncc1C(c1ccc(C(F)(F)F)cc1)N1CCc2ccccc2C1] | [symbol] | [symbol] |
| 7 | 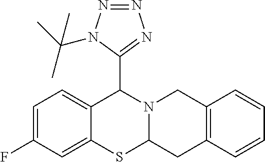 [SMILES: CC(C)(C)n1nnnc1C1c2ccc(F)cc2SC2Cc3ccccc3CN21] | [symbol] | [symbol] |
| 8 | 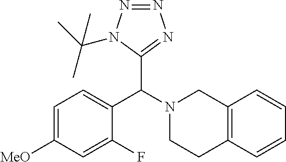 [SMILES: COc1ccc(C(c2nnnn2C(C)(C)C)N2CCc3ccccc3C2)c(F)c1] | [symbol] | [symbol] |
| 9 | 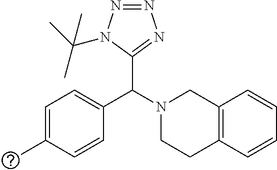 [SMILES: Cc1ccc(C(c2nnnn2C(C)(C)C)N2CCc3ccccc3C2)cc1] | [symbol] | [symbol] |
| 10 | 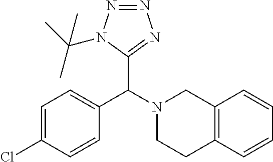 [SMILES: CC(C)(C)n1nnnc1C(c1ccc(Cl)cc1)N1CCc2ccccc2C1] | [symbol] | [symbol] |
| 11 | 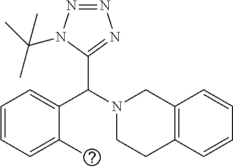 [SMILES: Cc1ccccc1C(c1nnnn1C(C)(C)C)N1CCc2ccccc2C1] | [symbol] | [symbol] |
| 12 | 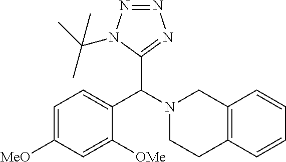 [SMILES: COc1ccc(C(c2nnnn2C(C)(C)C)N2CCc3ccccc3C2)c(OC)c1] | [symbol] | [symbol] |
| 13 | 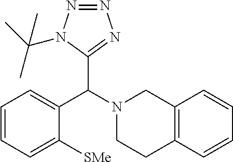 [SMILES: CSc1ccccc1C(c1nnnn1C(C)(C)C)N1CCc2ccccc2C1] | [symbol] | [symbol] |
| 14 | 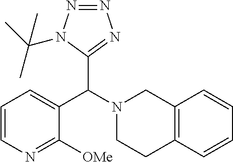 [SMILES: COc1ncccc1C(c1nnnn1C(C)(C)C)N1CCc2ccccc2C1] | [symbol] | [symbol] |
| 15 | 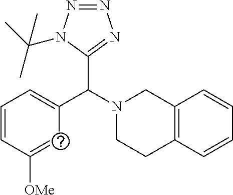 [SMILES: COc1cccc(C(c2nnnn2C(C)(C)C)N2CCc3ccccc3C2)c1] | [symbol] | [symbol] |
| 16 | 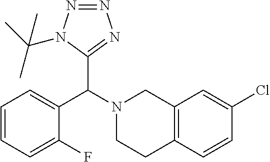 [SMILES: CC(C)(C)n1nnnc1C(c1ccccc1F)N1CCc2ccc(Cl)cc2C1] | [symbol] | [symbol] |
| 17 | 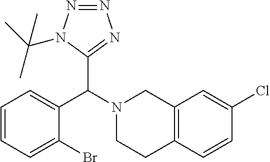 [SMILES: CC(C)(C)n1nnnc1C(c1ccccc1Br)N1CCc2ccc(Cl)cc2C1] | [symbol] | [symbol] |
| 18 | 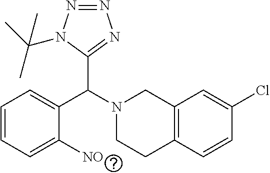 | [symbol] | [symbol] |
| 19 | 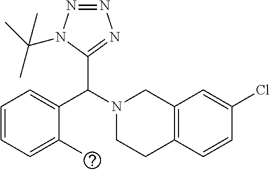 [SMILES: Cc1ccccc1C(c1nnnn1C(C)(C)C)N1CCc2ccc(Cl)cc2C1] | [symbol] | [symbol] |
| 20 | 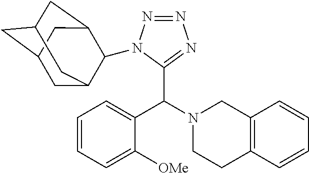 [SMILES: COc1ccccc1C(c1nnnn1C1C2CC3CC(C2)CC1C3)N1CCc2ccccc2C1] | [symbol] | [symbol] |
| 21 | 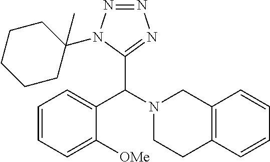 [SMILES: COc1ccccc1C(c1nnnn1C1(C)CCCCC1)N1CCc2ccccc2C1] | [symbol] | [symbol] |
| 22 | 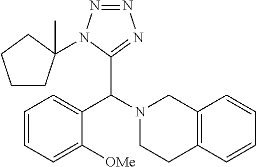 [SMILES: COc1ccccc1C(c1nnnn1C1(C)CCCC1)N1CCc2ccccc2C1] | [symbol] | [symbol] |
| 23 | 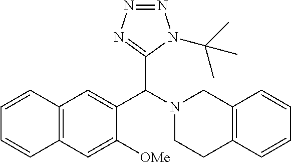 [SMILES: COc1cc2ccccc2cc1C(c1nnnn1C(C)(C)C)N1CCc2ccccc2C1] | [symbol] | [symbol] |
| 24 | 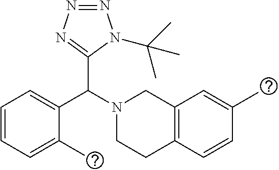 [SMILES: Cc1ccc2c(c1)CN(C(c1ccccc1C)c1nnnn1C(C)(C)C)CC2] | [symbol] | [symbol] |
| 25 | 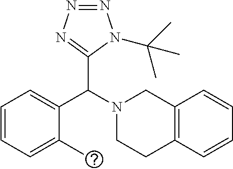 [SMILES: Cc1ccccc1C(c1nnnn1C(C)(C)C)N1CCc2ccccc2C1] | [symbol] | [symbol] |
| 26 | 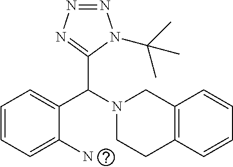 [SMILES: CC(C)(C)n1nnnc1C(c1ccccc1N)N1CCc2ccccc2C1] | [symbol] | [symbol] |
| 27 | 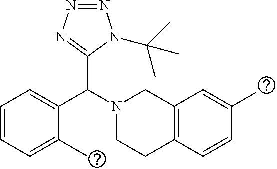 [SMILES: Cc1ccc2c(c1)CN(C(c1ccccc1C)c1nnnn1C(C)(C)C)CC2] | [symbol] | [symbol] |
| 28 | 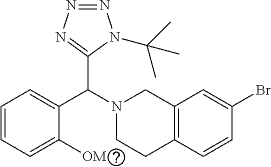 | [symbol] | [symbol] |
| 29 | 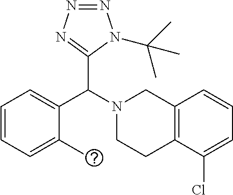 [SMILES: Cc1ccccc1C(c1nnnn1C(C)(C)C)N1CCc2c(Cl)cccc2C1] | [symbol] | [symbol] |
| 30 | 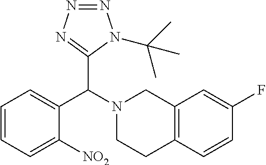 [SMILES: CC(C)(C)n1nnnc1C(c1ccccc1[N+](=O)[O-])N1CCc2ccc(F)cc2C1] | [symbol] | [symbol] |
| 31 | 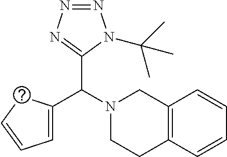 [SMILES: CC(C)(C)n1nnnc1C(C1=CC=CC1)N1CCc2ccccc2C1] | [symbol] | [symbol] |
| 32 | 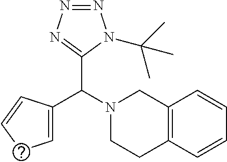 [SMILES: CC(C)(C)n1nnnc1C(C1=CCC=C1)N1CCc2ccccc2C1] | [symbol] | [symbol] |
| 33 | 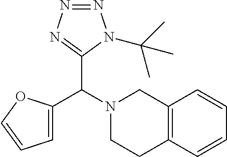 [SMILES: CC(C)(C)n1nnnc1C(c1ccco1)N1CCc2ccccc2C1] | [symbol] | [symbol] |
| 34 | 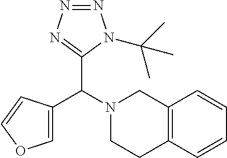 [SMILES: CC(C)(C)n1nnnc1C(c1ccoc1)N1CCc2ccccc2C1] | [symbol] | [symbol] |
| 35 | 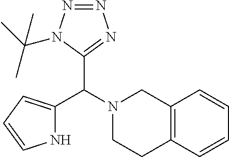 [SMILES: CC(C)(C)n1nnnc1C(c1ccc[nH]1)N1CCc2ccccc2C1] | [symbol] | [symbol] |
| 36 | 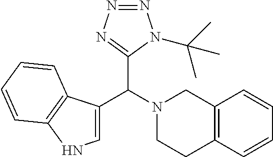 [SMILES: CC(C)(C)n1nnnc1C(c1c[nH]c2ccccc12)N1CCc2ccccc2C1] | [symbol] | [symbol] |
| 37 | 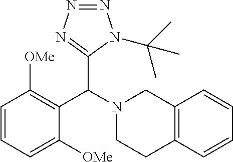 [SMILES: COc1cccc(OC)c1C(c1nnnn1C(C)(C)C)N1CCc2ccccc2C1] | [symbol] | [symbol] |
| 38 | 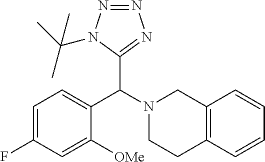 [SMILES: COc1cc(F)ccc1C(c1nnnn1C(C)(C)C)N1CCc2ccccc2C1] | [symbol] | [symbol] |
| 39 | 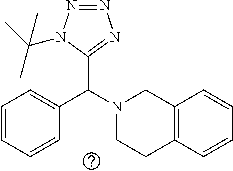 [SMILES: CC(C)(C)n1nnnc1C(c1ccccc1)N1CCc2ccccc2C1] | [symbol] | [symbol] |
| 40 | 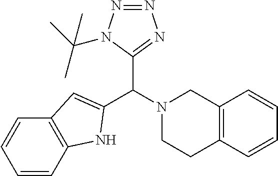 [SMILES: CC(C)(C)n1nnnc1C(c1cc2ccccc2[nH]1)N1CCc2ccccc2C1] | [symbol] | [symbol] |
| 41 | 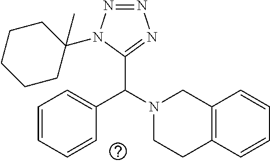 [SMILES: CC1(n2nnnc2C(c2ccccc2)N2CCc3ccccc3C2)CCCCC1] | [symbol] | [symbol] |
| 42 | 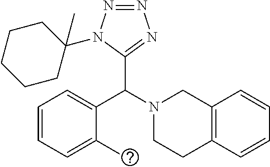 [SMILES: Cc1ccccc1C(c1nnnn1C1(C)CCCCC1)N1CCc2ccccc2C1] | [symbol] | [symbol] |
| 43 | 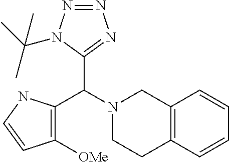 [SMILES: COc1cc[nH]c1C(c1nnnn1C(C)(C)C)N1CCc2ccccc2C1] | [symbol] | [symbol] |
| 44 | 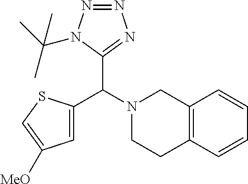 [SMILES: COc1csc(C(c2nnnn2C(C)(C)C)N2CCc3ccccc3C2)c1] | [symbol] | [symbol] |
| 45 | 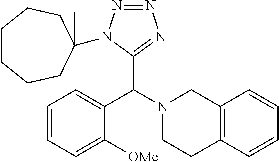 [SMILES: COc1ccccc1C(c1nnnn1C1(C)CCCCCC1)N1CCc2ccccc2C1] | [symbol] | [symbol] |
| 46 | 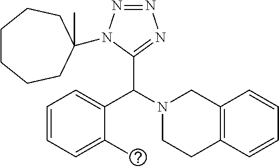 [SMILES: Cc1ccccc1C(c1nnnn1C1(C)CCCCCC1)N1CCc2ccccc2C1] | [symbol] | [symbol] |
| 47 | 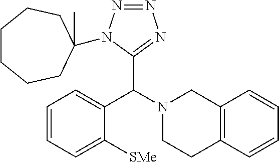 [SMILES: CSc1ccccc1C(c1nnnn1C1(C)CCCCCC1)N1CCc2ccccc2C1] | [symbol] | [symbol] |
| 48 | 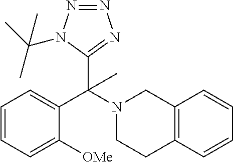 [SMILES: COc1ccccc1C(C)(c1nnnn1C(C)(C)C)N1CCc2ccccc2C1] | [symbol] | [symbol] |
| 49 | 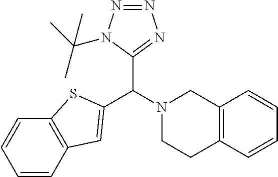 [SMILES: CC(C)(C)n1nnnc1C(c1cc2ccccc2s1)N1CCc2ccccc2C1] | [symbol] | [symbol] |
| 50 | 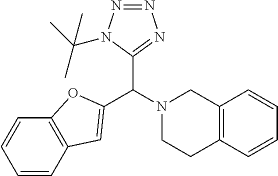 [SMILES: CC(C)(C)n1nnnc1C(c1cc2ccccc2o1)N1CCc2ccccc2C1] | [symbol] | [symbol] |
| 51 | 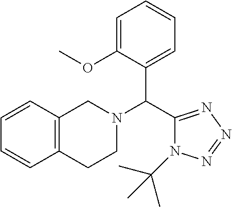 [SMILES: COc1ccccc1C(c1nnnn1C(C)(C)C)N1CCc2ccccc2C1] | [symbol] | [symbol] |
| 52 | 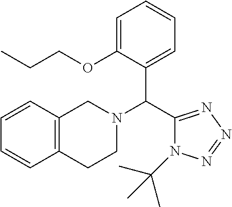 [SMILES: CCCOc1ccccc1C(c1nnnn1C(C)(C)C)N1CCc2ccccc2C1] | [symbol] | [symbol] |
| 53 | 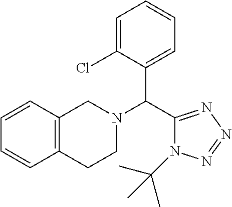 [SMILES: CC(C)(C)n1nnnc1C(c1ccccc1Cl)N1CCc2ccccc2C1] | [symbol] | [symbol] |
| 54 | 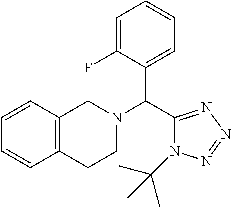 [SMILES: CC(C)(C)n1nnnc1C(c1ccccc1F)N1CCc2ccccc2C1] | [symbol] | [symbol] |
| 55 | 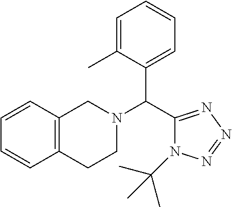 [SMILES: Cc1ccccc1C(c1nnnn1C(C)(C)C)N1CCc2ccccc2C1] | [symbol] | [symbol] |
| 56 | 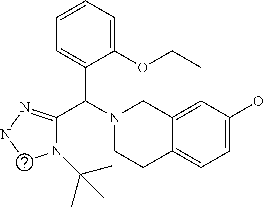 [SMILES: CCOc1ccccc1C(C1=NNCN1C(C)(C)C)N1CCc2ccc(O)cc2C1] | [symbol] | [symbol] |

[symbol]

indicates data missing or illegible when filed

### Example 57

### Synthesis of 2-((1-tert-butyl-1H-tetrazol-5-yl)(3,4-dihydroisoquinolin-2(1H)-yl)methyl)phenol

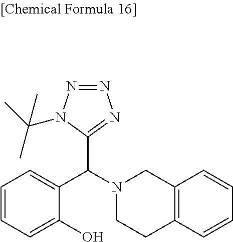 [SMILES: CC(C)(C)n1nnnc1C(c1ccccc1O)N1CCc2ccccc2C1]

To a 300-mL pressure-resistant glass container were added 45 mL of ethyl acetate, 0.45 g (1.0 mmol) of the compound of Example 1, and 0.11 g (10 wt %) of palladium on carbon. The contents of the container were stirred for 3 hours at room temperature in a hydrogen atmosphere (0.36 MPa). Filtration was then performed using celite, and the resultant solution was concentrated through use of an evaporator. The resultant residue was purified by silica gel column chromatography (n-hexane:AcOEt=3:1 v/v), and 0.33 g (89% yield) of the desired compound was obtained.

¹H-NMR (CDCl₃, δ) 1.75 (s, 9H), 2.88-2.95 (m, 2H), 3.06-3.15 (m, 1H), 3.27-3.35 (m, 1H), 3.68 (d, J=14.4 Hz, 1H), 4.21 (d, J=14.7 Hz, 1H), 6.02 (s, 1H), 6.24 (d, J=7.8 Hz, 1H), 6.75 (dt, J=1.2, 7.5 Hz, 1H), 6.94 (dd, J=1.2, 8.1 Hz, 2H), 7.08-7.16 (m, 3H), 7.24 (dt, J=1.2, 7.7 Hz, 1H)

### Example 58

### Synthesis of 2-((1-tert-butyl-1H-tetrazol-5-yl)(3,4-dihydroisoquinolin-2(1H)-yl)methyl)aniline

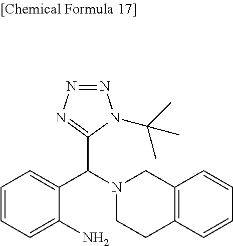 [SMILES: CC(C)(C)n1nnnc1C(c1ccccc1N)N1CCc2ccccc2C1]

To a 300-mL pressure-resistant glass container were added 100 mL of ethyl acetate, 0.5 g (1.27 mmol) of the compound of Example 11, and 0.1 g (10 wt %) of palladium on carbon. The contents of the container were stirred for 3 hours at room temperature in a hydrogen atmosphere (0.36 MPa). Filtration was then performed using celite, and the resultant solution was concentrated through use of an evaporator. The resultant residue was purified by silica gel column chromatography (n-hexane:AcOEt=2:1 v/v), and 0.39 g (85% yield) of the desired compound was obtained.

¹H-NMR (CDCl₃, δ) 1.64 (s, 9H), 2.81-3.25 (m, 4H), 3.70 (d, J=14.4 Hz, 1H), 4.24 (d, J=14.1 Hz, 1H), 4.71 (s, 2H), 5.87 (s, 1H), 6.39-7.18 (m, 8H)

### Examples 59-84

The compounds shown in Table 2 were created by the same procedure as in Example 1.

TABLE 2

| Ex- | | | | |
|---|---|---|---|---|
| ample | Structure | ¹H-NMR (400 MHz, CDCl3) δ | Name | MS |
| 59 | 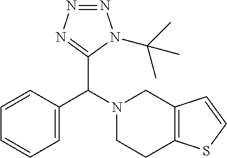 [SMILES: CC(C)(C)n1nnnc1C(c1ccccc1)N1CCc2sccc2C1] | 7.63(d, J = 8.4 Hz, 2H), 7.50-7.47(m, 3H), 7.43-7.32(m, 3H), 7.12-7.03(m, 6H), 6.91-6.85(m, 2H), 5.60 (s, 1H), 5.57(s, 1H), 4.06 (d, J = 14.4 Hz, 1H), 3.94 (d, J = 14.8 Hz, 1H), 3.71-3.64 (m, 2H), 3.09-3.02 (m, 3H), 2.83-2.69(m, 5H), 1.73(s, 9H), 1.34(s, 9H) | 5-((1-(tert-butyl)-1H-tetrazol-5- yl)(phenyl)methyl)-4,5,6,7- tetrahydrothieno[3,2-c]pyridine | 353.4 |
| 60 | 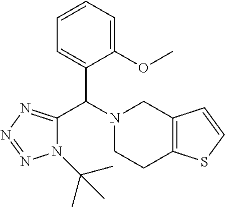 [SMILES: COc1ccccc1C(c1nnnn1C(C)(C)C)N1CCc2sccc2C1] | 7.68 (dd, J = 7.6, 1.8 Hz, 1H), 7.30 (dt, J = 10.1, 2.0 Hz, 1H), 7.01 (d, J = 4.8 Hz, 1H), 6.95 (q, J = 7.6 Hz, 2H), 6.63 (d, J = 5.2 Hz, 1H), 6.16 (s, 1H), 3.90 (s, 3H), 3.84 (d, J = 14.0 Hz, 1H), 3.67 (d, J = 14.4 Hz, 1H), 3.19 (q, J = 5.6 Hz, 1H), 2.88-2.81 (m, 3H), 1.67 (s, 9H) | 5-((1-(tert-butyl)-1H-tetrazol-5- yl)(2-methoxyphenyl)methyl)- 4,5,6,7-tetrahydrothieno[3,2- c]pyridine | 384.4 |
| 61 | 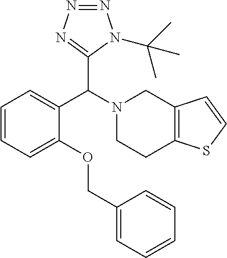 [SMILES: CC(C)(C)n1nnnc1C(c1ccccc1OCc1ccccc1)N1CCc2sccc2C1] | 7.66(dd, J = 7.6, 1.6 Hz, 1H), 7.43-7.40(m, 2H), 7.38- 7.28(m, 4H), 7.04-6.96(m, 3H), 6.62(d, J = 5.2 Hz, 1H), 6.22(s, 1H), 5.13(dd, J = 6.5, 6.0 Hz, 2H), 3.85(d, J = 14, 1H), 3.67(d, J = 14, 1H), 3.25-3.20(m, 1H), 2.92-2.86(m, 1H), 2.79(s 1H), 1.57(s, 9H) | 5-((2-(benzyloxy)phenyl)(1- (tert-butyl)-1H-tetrazol-5- yl)methyl)-4,5,6,7- tetrahydrothieno[3,2-c]pyridine | 450.8 |
| 62 | 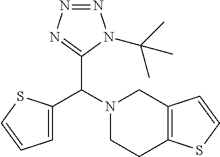 [SMILES: CC(C)(C)n1nnnc1C(c1cccs1)N1CCc2sccc2C1] | 7.36 (dd, J = 5.2, 1.4 Hz, 1H), 7.05 (d, J = 5.2 Hz, 1H), 6.98 (t, J = 4.4 Hz, 1H), 6.94-6.93 (m, 1H), 6.87 (d, J = 5.2 Hz, 1H), 5.89 (s, 1H), 3.88 (d, J = 13.6 Hz, 1H), 3.64 (d, J = 14.0 Hz, 1H), 3.20-3.14 (m, 1H), 2.99-2.94 (m, 1H), 2.90-2.77 (m, 2H), 1.76 (s, 9H) | 5-((1-(tert-butyl)-1H-tetrazol-5- yl)(thiophen-2-yl)methyl)- 4,5,6,7-tetrahydrothieno[3,2- c]pyridine | 360.2 |
| 63 | 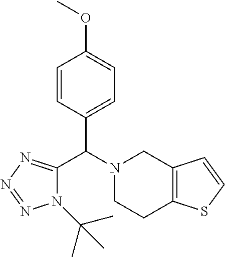 [SMILES: COc1ccc(C(c2nnnn2C(C)(C)C)N2CCc3sccc3C2)cc1] | 7.35 (d, J = 8.7 Hz, 2H), 7.04 (d, J = 5.1 Hz, 1H), 6.87 (d, J = 9.0 Hz, 2H), 6.63 (d, J = 5.1 Hz, 1H), 5.53 (s, 1H), 3.87 (d, J = 14.4 Hz, 1H), 3.80 (s, 3H), 3.56 (d, J = 14.1 Hz, 1H) 3.09-3.16 (m, 1H), 2.85-2.91 (m, 1H), 2.79-2.84 (m, 2H), 1.68 (s, 9H) | 5-((1-(tert-butyl)-1H-tetrazol-5- yl)(4-methoxyphenyl)methyl)- 4,5,6,7-tetrahydrothieno[3,2- c]pyridine | 384.4 |
| 64 | 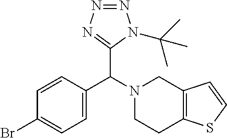 [SMILES: CC(C)(C)n1nnnc1C(c1ccc(Br)cc1)N1CCc2sccc2C1] | 7.50(d, J = 8.4 Hz, 2H), 7.33(d, J = 8.4 Hz, 2H), 7.05 (d, J = 5.1 Hz, 1H), 6.63 (d, J = 5.1 Hz, 1H), 5.57 (s, 1H), 3.87(d, J = 13.8 Hz, 1H), 3.55(d, J = 13.8 Hz, 1H), 3.15-3.08 (m, 1H), 2.90-2.78 (m, 3H), 1.70 (s, 9H) | 5-((4-bromophenyl)(1-(tert- butyl)-1H-tetrazol-5-yl)methyl)- 4,5,6,7-tetrahydrothieno[3,2- c]pyridine | 434.6 |
| 65 | 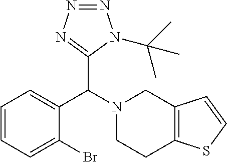 [SMILES: CC(C)(C)n1nnnc1C(c1ccccc1Br)N1CCc2sccc2C1] | 7.64(dd, J = 7.8 Hz, 1.5 Hz, 1H), 7.59(dd, J = 7.8 Hz, 1.5 Hz, 1H), 7.31 (ddd, J = 7.5 Hz, 1.2 Hz, 1H), 7.21 (ddd, J = 7.8 Hz, 7.8 Hz, 1.8 Hz, 1H), 7.03(d, J = 5.4 Hz, 1H), 6.65(d, J = 5.1 Hz, 1H), 6.15 (s, 1H), 4.00 (d, J = 14.1 Hz, 1H), 3.67 (d, J = 13.8 Hz, 1H), 3.24-3.16 (m, 1H), 3.02-2.95 (m, 1H), 2.85-2.69(m, 2H), 1.71 (s, 9H) | 5-((2-bromophenyl)(1-(tert- butyl)-1H-tetrazol-5-yl)methyl)- 4,5,6,7-tetrahydrothieno[3,2- c]pyridine | 432.6 |
| 66 | 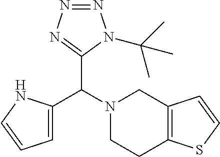 [SMILES: CC(C)(C)n1nnnc1C(c1ccc[nH]1)N1CCc2sccc2C1] | 9.805 (s, 1H), 7.035 (d, J = 5.2, 1H), 6.861-6.844 (m, 1H), 6.651 (d, J = 5.2, 1H), 6.192-6.174 (m, 1H), 6.149- 6.134 (m, 1H), 5.519 (s, 1H), 3.688 (d, J = 14.4, 1H), 3.510 (d, J = 14, 1H), 3.064-3.011 (m, 1H), 2.890-2.824 (m, 1H), 2.775-2.659 (m, 2H), 1.776 (s, 9H) | 5-((1-(tert-butyl)-1H-tetrazol-5- yl)(1H-pyrrol-2-yl)methyl)- 4,5,6,7-tetrahydrothieno[3,2- c]pyridine | 343.2 |
| 67 | 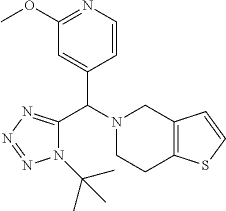 [SMILES: COc1cc(C(c2nnnn2C(C)(C)C)N2CCc3sccc3C2)ccn1] | 7.26 (t, J = 8.0 Hz, 1H), 7.05 (s, 1H), 7.04 (d, J = 2.8 Hz, 1H), 6.93 (d, J = 7.6 Hz, 1H), 6.88 (dd, J = 2.8 Hz, 1H), 8.63 (d, J = 7.6 Hz, 1H), 5.56 (s, 1H), 3.59 (d, J = 13.2 Hz, 1H), 3.79 (s, 3H), 3.58 (d, J = 14.0 Hz, 1H), 3.18-3.12 (m, 1H), 2.92-2.88 (m, 1H), 2.82 (t, J = 5.2 Hz, 2H), 1.69 (s, 9H) | 5-((1-(tert-butyl)-1H-tetrazol-5- yl)(3-methoxyphenyl)methyl)- 4,5,6,7-tetrahydrothieno[3,2- c]pyridine | 384.6 |
| 68 | 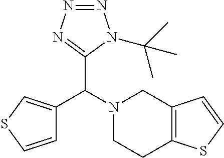 [SMILES: CC(C)(C)n1nnnc1C(c1ccsc1)N1CCc2sccc2C1] | 7.354-7.285 (m, 2H), 7.243-7.233 (m, 1H), 7.045 (d, J = 4.8, 1H), 6.657 (d, J = 5.2, 1H), 5.693 (s, 1H), 3.850 (d, J = 14, 1H), 3.594 (d, J = 14, 1H), 3.167-3.110 (m, 1H), 2.902-2.773 (m, 3H), 1.732 (s, 9H) | 5-((1-(tert-butyl)-1H-tetrazol-5- yl)(thiophen-3-yl)methyl)- 4,5,6,7-tetrahydrothieno[3,2- c]pyridine | 360.4 |
| 69 | 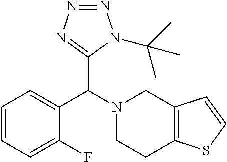 [SMILES: CC(C)(C)n1nnnc1C(c1ccccc1F)N1CCc2sccc2C1] | 7.88-7.84 (ddd, J = 7.6, 7.6, 1.6 Hz, 1H), 7.37-7.31 (m, 1H), 7.20-7.16 (ddd, J = 7.6, 7.6, 1.2 Hz, 1H), 7.14-7.09 (m, 1H), 7.04-7.03 (d, J = 5.2 Hz, 1H), 6.66-6.64 (d, J = 5.2 Hz, 1H), 6.02 (s, 1H), 3.84-3.81 (d, J = 14 Hz, 1H), 3.71-3.67 (d, J = 14 Hz, 1H), 3.25-3.18 (m, 1H), 2.88- 2.78 (m, 3H), 1.72 (s, 9H) | 5-((1-(tert-butyl)-1H-tetrazol-5- yl)(2-fluorophenyl)methyl)- 4,5,6,7-tetrahydrothieno[3,2- c]pyridine | 372.4 |
| 70 | 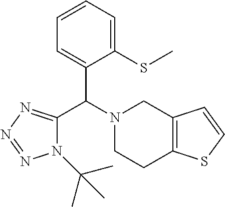 [SMILES: CSc1ccccc1C(c1nnnn1C(C)(C)C)N1CCc2sccc2C1] | 7.41-7.39 (dd, J = 8.0, 1.2 Hz, 1H), 7.34-7.30 (ddd, J = 7.2, 7.2, 2.0 Hz, 1H), 7.12-7.04 (m, 2H), 7.02-7.00 (d, J = 5.2 Hz, 1H), 6.65-6.63 (d, J = 5.2 Hz, 1H), 6.19 (s, 1H), 4.18-4.14 (d, J = 13.6 Hz, 1H), 3.57-3.54 (d, J = 14 Hz, 1H), 3.27-3.21 (m, 1H), 3.03-2.97 (m, 1H), 2.83-2.66 (m, 2H), 2.50 (s, 3H), 1.64 (s, 9H) | 5-((1-(tert-butyl)-1H-tetrazol-5- yl)(2- (methylthio)phenyl)methyl)- 4,5,6,7-tetrahydrothieno[3,2- c]pyridine | 400.4 |
| 71 | 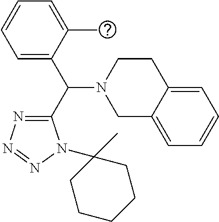 [SMILES: Cc1ccccc1C(c1nnnn1C1(C)CCCCC1)N1CCc2ccccc2C1] | 7.25 (dt, J = 1.4, 7.7 Hz, 1H), 7.16-7.07 (m, 3H), 6.93 (d, J = 8.1 Hz, 2H), 6.74 (d, J = 1.2, 7.6 Hz, 1H), 6.30 (d, J = 7.3 Hz, 1H), 6.01 (s, 1H), 4.19 (d, J = 14.4 Hz, 1H), 3.66 (d, J = 14.4 Hz, 1H), 3.32-3.24 (m, 1H), 3.11- 3.04 (m, 1H), 2.94-2.87 (m, 2H), 2.55-2.48 (m, 1H), 2.31-2.25 (m, 1H), 2.09-2.01 (m, 1H), 1.90-1.82 (m, 1H), 1.68-1.65 (m, 2H), 1.59 (s, 3H), 1.50-1.46 (m, 4H) | 2-((3,4-dihydroisoquinolin- 2(1H)-yl)-(1-(1- methylcyclohexyl)-1H-tetrazol- 5-yl)methyl)phenol | 403.2 |
| 72 | 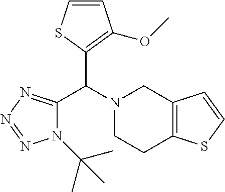 [SMILES: COc1ccsc1C(c1nnnn1C(C)(C)C)N1CCc2sccc2C1] | 7.28 (d, J = 5.6 Hz, 1H), 7.01 (d, J = 5.1 Hz, 1H), 6.85 (d, J = 5.6 Hz, 1H), 6.68 (d, J = 5.1 Hz, 1H), 6.11 (s, 1H), 3.88 (s, 3H), 3.79 (d, J = 14.4 Hz, 1H), 3.65 (d, J = 14.3 Hz, 1H), 3.14-3.06 (m, 1H), 2.95-2.83 (m, 2H), 2.79-2.72 (m, 1H), 1.76 (s, 9H). | 5-((1-(tert-butyl)-1H-tetrazol-5- yl)(3-methoxythiophen-2- yl)methyl)-4,5,6,7- tetrahydrothieno[3,2-c]pyridine | 390.0 |
| 73 | 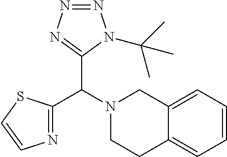 [SMILES: CC(C)(C)n1nnnc1C(c1nccs1)N1CCc2ccccc2C1] | 7.78 (d, J = 3.3 Hz, 1H), 7.46 (d, J = 3.0 Hz, 1H), 7.11- 7.04 (m, 3H), 6.97-6.94 (m, 1H), 6.10 (s, 1H), 3.85 (d, J = 4.5 Hz, 2H), 3.10-3.02 (m, 1H), 2.97-2.75 (m, 3H), 1.80 (s, 9H). | 2-((1-(tert-butyl)-1H-tetrazol-5- yl)(3,4-dihydroisoquinolin- 2(1H)-yl)methyl)thiazole | 354.6 |
| 74 | 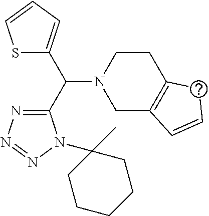 [SMILES: CC1(n2nnnc2C(c2cccs2)N2CCC3=C(C=CC3)C2)CCCCC1] | 7.35 (dd, J = 2.2, 4.1 Hz, 1H), 7.03 (d, J = 6.2 Hz 1H), 6.97-6.96 (m, 2H), 6.65 (d, J = 5.1 Hz, 1H), 5.85 (s, 1H), 3.88 (d, J = 14.0 Hz, 1H), 3.64 (d, J = 14.0 Hz, 1H), 3.18-3.11 (m, 1H), 2.98-2.91 (m, 1H), 2.85-2.76 (m, 2H), 2.73-2.55 (m, 1H), 2.49-2.42 (m, 1H), 1.95- 1.82 (m, 2H), 1.62-1.51 (m, 5H), 1.50-1.38 (m, 4H). | 5-((1-(1-methylcyclohexyl)-1H- tetrazol-5-yl)(thiophen-2- yl)methyl)-4,5,6,7- tetrahydrothieno[3,2-c]pyridine | 399.6 |
| 75 | 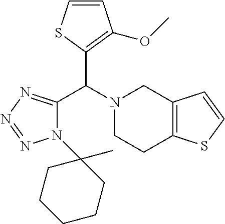 [SMILES: COc1ccsc1C(c1nnnn1C1(C)CCCCC1)N1CCc2sccc2C1] | 7.27 (d, J = 5.7 Hz, 1H), 7.01 (d, J = 5.1 Hz, 1H), 8.84 (d, J = 6.7 Hz, 1H), 6.65 (d, J = 5.1 Hz, 1H), 6.04 (s, 1H), 3.88 (s, 3H), 3.79 (d, J = 14.4 Hz, 1H), 3.63 (d, J = 14.1 Hz, 1H), 3.10-3.04 (m, 1H), 2.94-2.84 (m, 2H), 2.79-2.56 (m, 3H), 1.89-1.81 (m, 2H), 1.60-1.51 (m, 5H), 1.48-1.40 (m, 4H). | 5-((3-methoxythiophen-2-yl)(1- (1-methylcyclohexyl)-1H- tetrazol-5-yl)methyl)-4,5,6,7- tetrahydrothieno[3,2-c]pyridine | 429.8 |
| 76 | 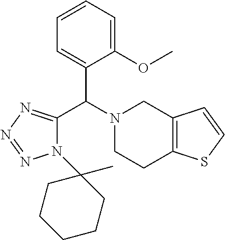 [SMILES: COc1ccccc1C(c1nnnn1C1(C)CCCCC1)N1CCc2sccc2C1] | 7.63 (dd, J = 1.7, 7.6 Hz, 1H), 7.30 (dt, J = 1.7, 7.8 Hz, 1H), 7.01 (d, J = 5.1 Hz, 1H), 6.98-6.92 (m, 2H), 6.63 (d, J = 5.1 Hz, 1H), 6.09 (s, 1H), 3.90 (s, 3H), 3.83 (d, J = 14.3 Hz, 1H), 3.69 (d, J = 14.2 Hz, 1H), 3.22-3.15 (m, 1H), 2.92-2.81 (m, 3H), 2.85-2.57 (m, 1H), 2.49- 2.42 (m, 1H), 1.84-1.70 (m, 3H), 1.61-1.49 (m, 2H), 1.43-1.37 (m, 6H). | 5-((2-methoxyphenyl)(1-(1- methylcyclohexyl)-1H-tetrazol- 5-yl)methyl)-4,5,6,7- tetrahydrothieno[3,2-c]pyridine | 424.0 |
| 77 | 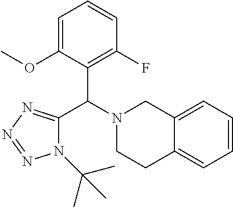 [SMILES: COc1cccc(F)c1C(c1nnnn1C(C)(C)C)N1CCc2ccccc2C1] | 1H NMR (300 MHz, CDCl3) δ 7.36-7.28(m, 2H), 7.08- 6.73(m, 6H), 6.04(s, 1H), 3.86(s, 5H), 3.42-3.39(m, 1H), 2.96-2.82(m, 3H), 1.56(s, 9H) | 2-((1-(tert-butyl)-1H-tetrazol-5- yl)(2-fluoro-6- methoxyphenyl)methyl)- 1,2,3,4-tetrahydroisoquinoline | 396.8 |
| 78 | 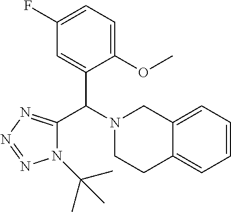 [SMILES: COc1ccc(F)cc1C(c1nnnn1C(C)(C)C)N1CCc2ccccc2C1] | 7.63-7.59(m, 1H), 7.09-6.85(m, 7H), 6.13(s, 1H), 3.90- 3.85(m, 3H), 3.74(d, J = 14.1 Hz, 1H), 3.11-3.09(m, 1H), 2.85-2.78(m, 3H), 1.73(s, 9H) | 2-((1-(tert-butyl)-1H-tetrazol-5- yl)(5-fluoro-2- methoxyphenyl)methyl)- 1,2,3,4-tetrahydroisoquinoline | 397.0 |
| 79 | 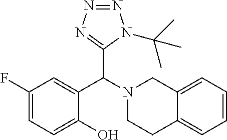 [SMILES: CC(C)(C)n1nnnc1C(c1cc(F)ccc1O)N1CCc2ccccc2C1] | 7.14-6.90(m, 7H), 5.98(s, 1H), 4.20(d, J = 15 Hz, 1H), 3.66(d, J = 15 Hz, 1H), 3.40-2.91(m, 4H), 1.76(s, 9H) | 2-((1-(tert-butyl)-1H-tetrazol-5- yl)(3,4-dihydroisoquinolin- 2(1H)-yl)methyl)-4- fluorophenol | 383.0 |
| 80 | 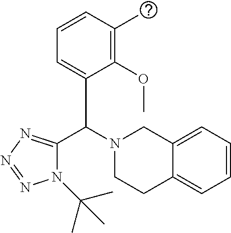 [SMILES: COc1c(C)cccc1C(c1nnnn1C(C)(C)C)N1CCc2ccccc2C1] | 7.37-6.93(m, 7H), 6.21(s, 1H), 4.06-4.00(m, 4H), 3.74(d, J = 15 Hz, 1H), 2.93-2.80(m, 4H), 1.70(s, 9H) | 2-((1-(tert-butyl)-1H-tetrazol-5- yl)(3-fluoro-2- methoxyphenyl)methyl)- 1,2,3,4-tetrahydroisoquinoline | 396.8 |
| 81 | 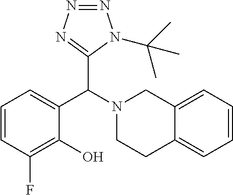 [SMILES: CC(C)(C)n1nnnc1C(c1cccc(F)c1O)N1CCc2ccccc2C1] | 7.16-6.66(m, 7H), 8.05(s, 2H), 4.21(d, J = 15 Hz, 1H), 3.69(d, J = 15 Hz, 1H), 3.35-2.91(m, 4H), 1.76(s, 9H) | 2-((1-(tert-butyl)-1H-tetrazol-5- yl)(3,4-dihydroisoquinolin- 2(1H)-yl)methyl)-5- fluorophenol | 383.0 |
| 82 | 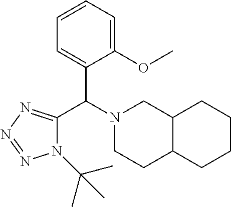 [SMILES: COc1ccccc1C(c1nnnn1C(C)(C)C)N1CCC2CCCCC2C1] | 7.70-7.66(m, 1H), 7.29-7.25(m, 1H), 6.95-6.89(m, 2H), 5.88(d, J = 12.0 Hz, 1H), 3.87(s, 3H), 3.87-2.66(m, 2H), 2.36-0.82(m, 23H) | 2-((1-(tert-butyl)-1H-tetrazol-5- yl)(2- methoxyphenyl)methyl)deca- hydroisoquinoline | 383.8 |
| 83 | 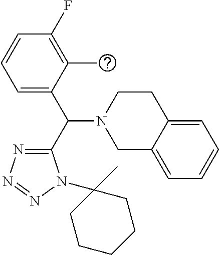 [SMILES: Cc1c(F)cccc1C(c1nnnn1C1(C)CCCCC1)N1CCc2ccccc2C1] | 7.16-6.66(m, 7H), 6.05(s, 1H), 4.21(d, J = 15 Hz, 1H), 3.69(d, J = 15 Hz, 1H), 3.35-2.86(m, 2H), 2.57-2.02(m, 3H), 1.88-1.60(m, 13H) | 2-((3,4-dihydroisoquinolin- 2(1H)-yl)(1-(1- methylcylohexyl)-1H-tetrazol- 5-yl)(methyl)-6-fluorophenol | 421.5 |

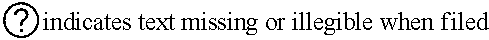

### 2. Evaluation of Pharmacological Activity

The positive allosteric regulation activity with respect to PGI₂ receptors (IP) of the tetrazole derivative as the active ingredient of the present invention was evaluated as described in Test Examples 1 through 4 (Methods A through D) and Test Example 5 (Magnus test).

### Test Example 1

### Method A

Evaluation of Positive Allosteric Modulator Activity on PGI₂ Receptors

The positive allosteric modulator activity of a test compound was measured as a change in the amount of intracellular cyclic AMP (cAMP) in the presence of 100 pM of the IP agonist iloprost (manufactured by CAYMAN CHEMICAL), using CHO-K1 cells (CHO/hIP cells) in which human PGI₂ receptors (hIP) are stably expressed.

An amount of 5 μL of a mixture (diluted in F-12 culture medium (manufactured by GIBCO) including 0.1% BSA) of iloprost dissolved in DMSO and a test compound dissolved in DMSO was dispensed in advance into a 384-well black microplate, and 5 μL of CHO/hIP cells suspended at a concentration of 1.6×10⁶ cells/mL in F-12 culture medium including 0.1% BSA were added to the microplate. After 40 minutes of incubation at room temperature, the amount of cAMP was measured using a cAMP assay kit (cAMP HiRange, manufactured by Cisbio Bioassays). Specifically, 5 μL of a cAMP-d2 solution (including a cell lysate) from the kit were added and the reaction was stopped, then 5 μL of a fluorescence-labeled cAMP antibody solution from the kit was added and incubation was performed for 1 hour at room temperature, after which fluorescence was measured using a PHERAstar plate reader (manufactured by BMG) in HTRF (registered trademark) mode. The amount of cAMP in each well was calculated from a cAMP calibration curve obtained using a standard sample, and the activity of each test compound is noted below as a ratio T/C (%) based on the amount of cAMP for 100 pM of isoprost alone. Results are shown in Table 3. All of the compounds included in this table were purchased from Asinex Corporation.

TABLE 3

| Compound | Test method | T/C % | [symbol] |
|---|---|---|---|
| 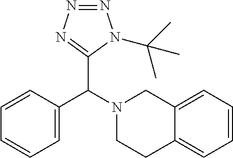 [SMILES: CC(C)(C)n1nnnc1C(c1ccccc1)N1CCc2ccccc2C1] | Method A | 143 | 5 uM |
| 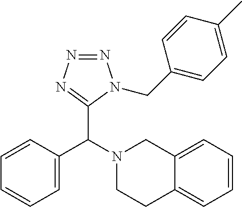 [SMILES: Cc1ccc(Cn2nnnc2C(c2ccccc2)N2CCc3ccccc3C2)cc1] | Method A | 117 | 10 uM |
| 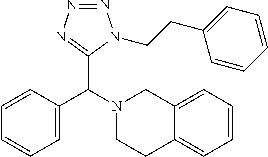 [SMILES: c1ccc(CCn2nnnc2C(c2ccccc2)N2CCc3ccccc3C2)cc1] | Method A | 128 | 10 uM |
| 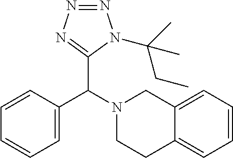 [SMILES: CCC(C)(C)n1nnnc1C(c1ccccc1)N1CCc2ccccc2C1] | Method A | 202 | 10 uM |
| 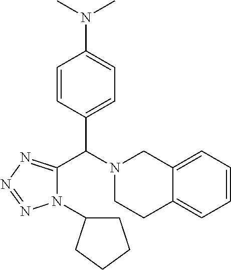 [SMILES: CN(C)c1ccc(C(c2nnnn2C2CCCC2)N2CCc3ccccc3C2)cc1] | Method A | 103 | 10 uM |
| 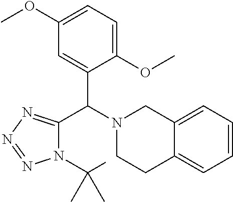 [SMILES: COc1ccc(OC)c(C(c2nnnn2C(C)(C)C)N2CCc3ccccc3C2)c1] | Method A | 100 | 10 uM |
| 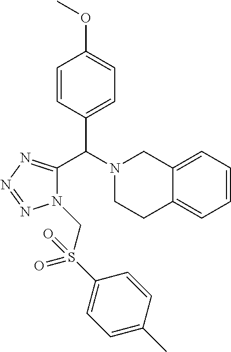 [SMILES: COc1ccc(C(c2nnnn2CS(=O)(=O)c2ccc(C)cc2)N2CCc3ccccc3C2)cc1] | Method A | 101 | 10 uM |
| 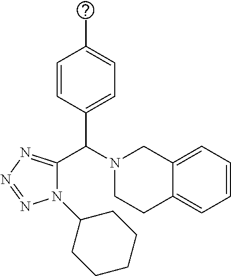 [SMILES: Cc1ccc(C(c2nnnn2C2CCCCC2)N2CCc3ccccc3C2)cc1] | Method A | 100 | 10 uM |
| 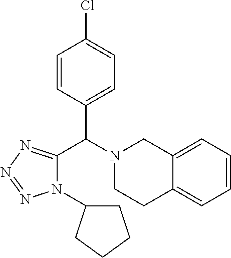 [SMILES: Clc1ccc(C(c2nnnn2C2CCCC2)N2CCc3ccccc3C2)cc1] | Method A | 110 | 10 uM |
| 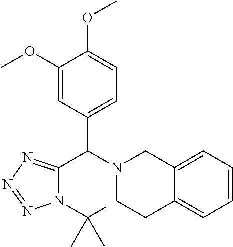 [SMILES: COc1ccc(C(c2nnnn2C(C)(C)C)N2CCc3ccccc3C2)cc1OC] | Method A | 103 | 10 uM |
| 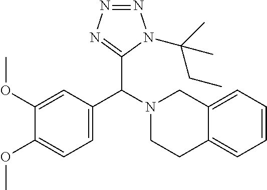 [SMILES: CCC(C)(C)n1nnnc1C(c1ccc(OC)c(OC)c1)N1CCc2ccccc2C1] | Method A | 103 | 10 uM |
| 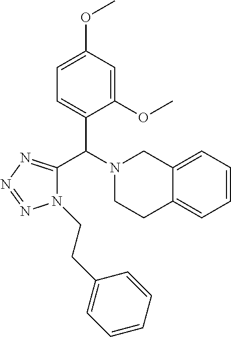 [SMILES: COc1ccc(C(c2nnnn2CCc2ccccc2)N2CCc3ccccc3C2)c(OC)c1] | Method A | 106 | 10 uM |
| 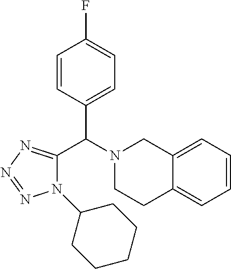 [SMILES: Fc1ccc(C(c2nnnn2C2CCCCC2)N2CCc3ccccc3C2)cc1] | Method A | 104 | 10 uM |
| 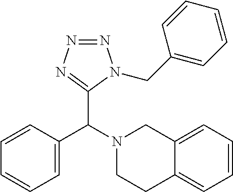 [SMILES: c1ccc(Cn2nnnc2C(c2ccccc2)N2CCc3ccccc3C2)cc1] | Method A | 112 | 10 uM |
| 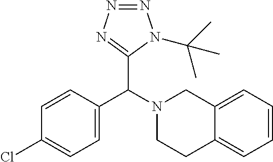 [SMILES: CC(C)(C)n1nnnc1C(c1ccc(Cl)cc1)N1CCc2ccccc2C1] | Method A | 113 | 5 uM |
| 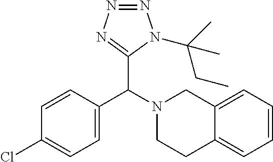 [SMILES: CCC(C)(C)n1nnnc1C(c1ccc(Cl)cc1)N1CCc2ccccc2C1] | Method A | 148 | 10 uM |
| 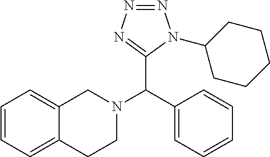 [SMILES: c1ccc(C(c2nnnn2C2CCCCC2)N2CCc3ccccc3C2)cc1] | Method A | 112 | 10 uM |
| 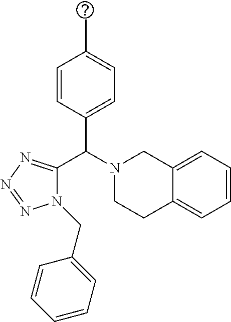 [SMILES: Cc1ccc(C(c2nnnn2Cc2ccccc2)N2CCc3ccccc3C2)cc1] | Method A | 112 | 10 uM |
| 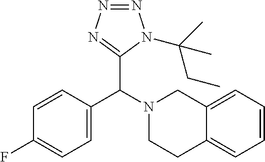 [SMILES: CCC(C)(C)n1nnnc1C(c1ccc(F)cc1)N1CCc2ccccc2C1] | Method A | 155 | 10 uM |
| 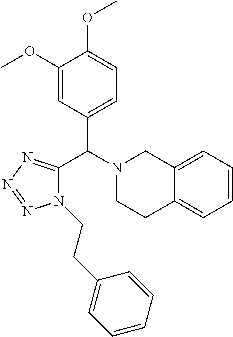 [SMILES: COc1ccc(C(c2nnnn2CCc2ccccc2)N2CCc3ccccc3C2)cc1OC] | Method A | 106 | 10 uM |
| 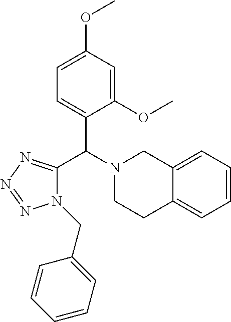 [SMILES: COc1ccc(C(c2nnnn2Cc2ccccc2)N2CCc3ccccc3C2)c(OC)c1] | Method A | 103 | 10 uM |
| 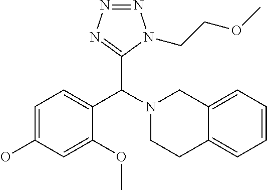 [SMILES: COCCn1nnnc1C(c1ccc(O)cc1OC)N1CCc2ccccc2C1] | Method A | 97 | 10 uM |
| 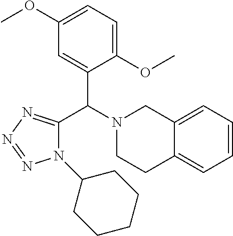 [SMILES: COc1ccc(OC)c(C(c2nnnn2C2CCCCC2)N2CCc3ccccc3C2)c1] | Method A | 82 | 10 uM |
| 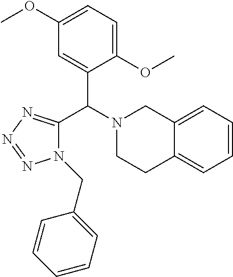 [SMILES: COc1ccc(OC)c(C(c2nnnn2Cc2ccccc2)N2CCc3ccccc3C2)c1] | Method A | 90 | 10 uM |
| 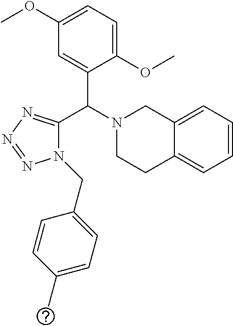 [SMILES: COc1ccc(OC)c(C(c2nnnn2Cc2ccc(C)cc2)N2CCc3ccccc3C2)c1] | Method A | 91 | 10 uM |
| 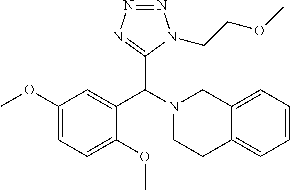 [SMILES: COCCn1nnnc1C(c1cc(OC)ccc1OC)N1CCc2ccccc2C1] | Method A | 90 | 10 uM |
| 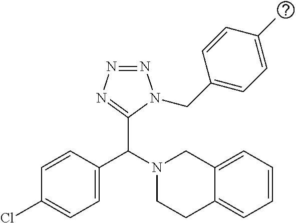 [SMILES: Cc1ccc(Cn2nnnc2C(c2ccc(Cl)cc2)N2CCc3ccccc3C2)cc1] | Method A | 109 | 10 uM |
| 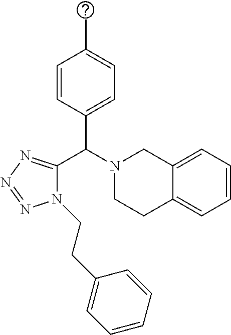 [SMILES: Cc1ccc(C(c2nnnn2CCc2ccccc2)N2CCc3ccccc3C2)cc1] | Method A | 108 | 10 uM |
| 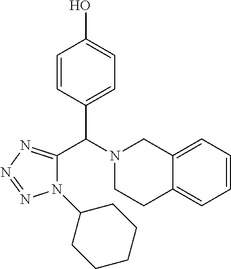 [SMILES: Oc1ccc(C(c2nnnn2C2CCCCC2)N2CCc3ccccc3C2)cc1] | Method A | 104 | 10 uM |
| 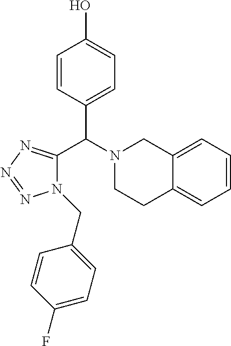 [SMILES: Oc1ccc(C(c2nnnn2Cc2ccc(F)cc2)N2CCc3ccccc3C2)cc1] | Method A | 113 | 10 uM |
| 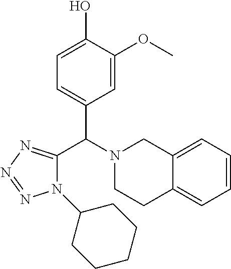 [SMILES: COc1cc(C(c2nnnn2C2CCCCC2)N2CCc3ccccc3C2)ccc1O] | Method A | 97 | 10 uM |
| 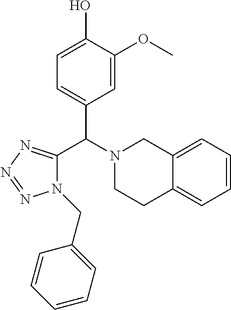 [SMILES: COc1cc(C(c2nnnn2Cc2ccccc2)N2CCc3ccccc3C2)ccc1O] | Method A | 94 | 10 uM |
| 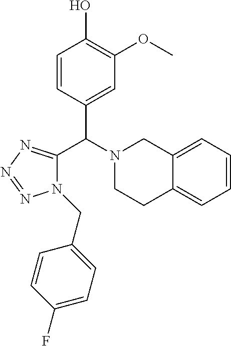 [SMILES: COc1cc(C(c2nnnn2Cc2ccc(F)cc2)N2CCc3ccccc3C2)ccc1O] | Method A | 91 | 10 uM |
| 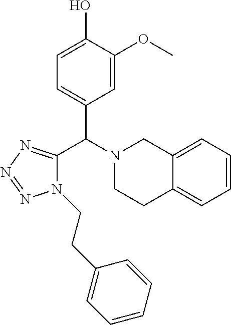 [SMILES: COc1cc(C(c2nnnn2CCc2ccccc2)N2CCc3ccccc3C2)ccc1O] | Method A | 91 | 10 uM |
| 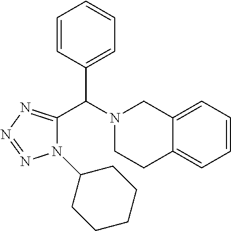 [SMILES: c1ccc(C(c2nnnn2C2CCCCC2)N2CCc3ccccc3C2)cc1] | Method A | 98 | 10 uM |
| 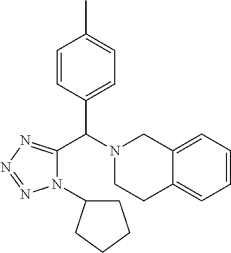 [SMILES: Cc1ccc(C(c2nnnn2C2CCCC2)N2CCc3ccccc3C2)cc1] | Method A | 105 | 10 uM |
| 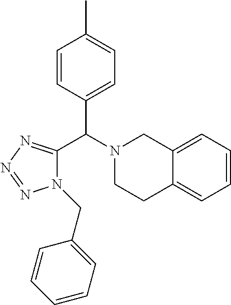 [SMILES: Cc1ccc(C(c2nnnn2Cc2ccccc2)N2CCc3ccccc3C2)cc1] | Method A | 102 | 10 uM |
| 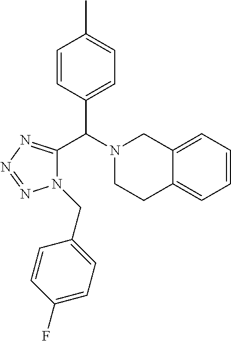 [SMILES: Cc1ccc(C(c2nnnn2Cc2ccc(F)cc2)N2CCc3ccccc3C2)cc1] | Method A | 117 | 10 uM |
| 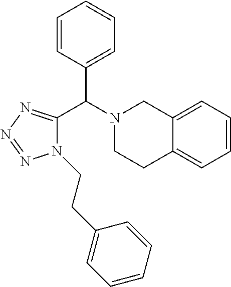 [SMILES: c1ccc(CCn2nnnc2C(c2ccccc2)N2CCc3ccccc3C2)cc1] | Method A | 111 | 10 uM |
| 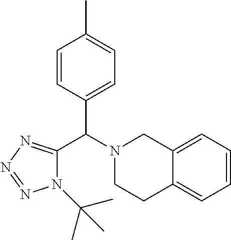 [SMILES: Cc1ccc(C(c2nnnn2C(C)(C)C)N2CCc3ccccc3C2)cc1] | Method A | 112 | 5 uM |
| 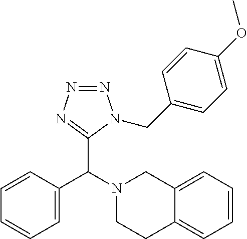 [SMILES: COc1ccc(Cn2nnnc2C(c2ccccc2)N2CCc3ccccc3C2)cc1] | Method A | 104 | 10 uM |
| 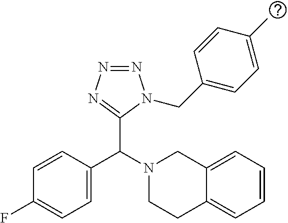 [SMILES: Cc1ccc(Cn2nnnc2C(c2ccc(F)cc2)N2CCc3ccccc3C2)cc1] | Method A | 106 | 10 uM |
| 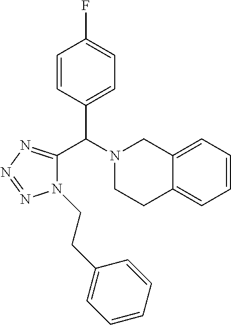 [SMILES: Fc1ccc(C(c2nnnn2CCc2ccccc2)N2CCc3ccccc3C2)cc1] | Method A | 103 | 10 uM |
| 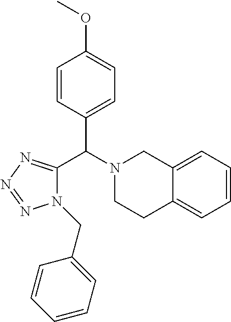 [SMILES: COc1ccc(C(c2nnnn2Cc2ccccc2)N2CCc3ccccc3C2)cc1] | Method A | 110 | 10 uM |
| 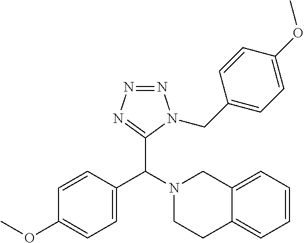 [SMILES: COc1ccc(Cn2nnnc2C(c2ccc(OC)cc2)N2CCc3ccccc3C2)cc1] | Method A | 105 | 10 uM |
| 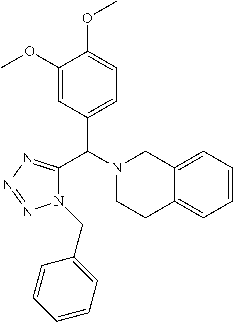 [SMILES: COc1ccc(C(c2nnnn2Cc2ccccc2)N2CCc3ccccc3C2)cc1OC] | Method A | 90 | 5 uM |
| 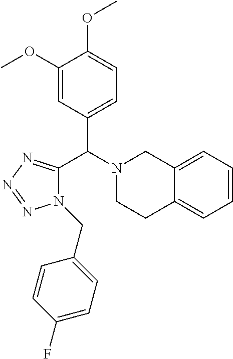 [SMILES: COc1ccc(C(c2nnnn2Cc2ccc(F)cc2)N2CCc3ccccc3C2)cc1OC] | Method A | 96 | 10 uM |
| 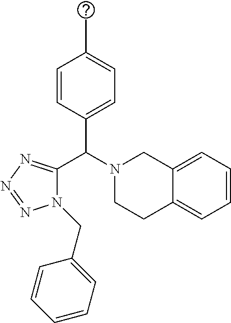 [SMILES: Cc1ccc(C(c2nnnn2Cc2ccccc2)N2CCc3ccccc3C2)cc1] | Method A | 103 | 10 uM |
| 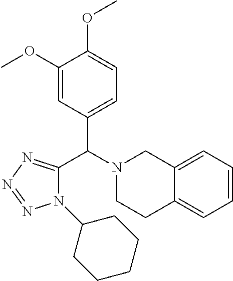 [SMILES: COc1ccc(C(c2nnnn2C2CCCCC2)N2CCc3ccccc3C2)cc1OC] | Method A | 87 | 10 uM |
| 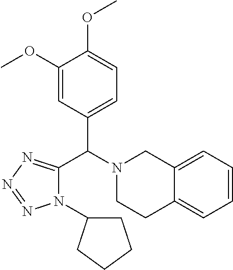 [SMILES: COc1ccc(C(c2nnnn2C2CCCC2)N2CCc3ccccc3C2)cc1OC] | Method A | 93 | 10 uM |
| 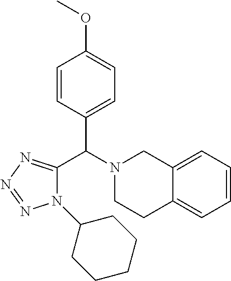 [SMILES: COc1ccc(C(c2nnnn2C2CCCCC2)N2CCc3ccccc3C2)cc1] | Method A | 111 | 10 uM |
| 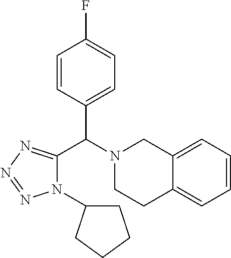 [SMILES: Fc1ccc(C(c2nnnn2C2CCCC2)N2CCc3ccccc3C2)cc1] | Method A | 108 | 10 uM |
| 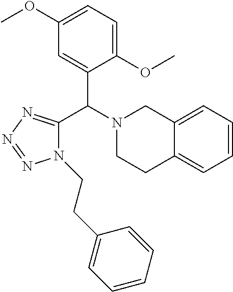 [SMILES: COc1ccc(OC)c(C(c2nnnn2CCc2ccccc2)N2CCc3ccccc3C2)c1] | Method A | 78 | 10 uM |
| 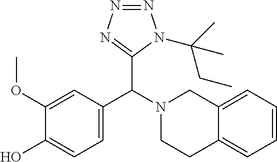 [SMILES: CCC(C)(C)n1nnnc1C(c1ccc(O)c(OC)c1)N1CCc2ccccc2C1] | Method A | 122 | 10 uM |
| 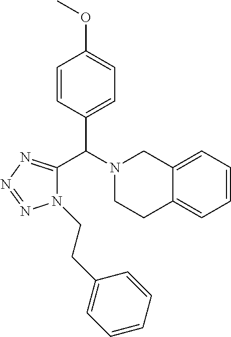 [SMILES: COc1ccc(C(c2nnnn2CCc2ccccc2)N2CCc3ccccc3C2)cc1] | Method A | 114 | 10 uM |
| 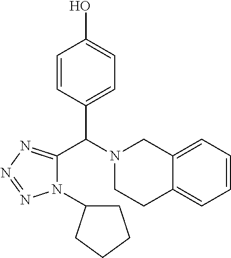 [SMILES: Oc1ccc(C(c2nnnn2C2CCCC2)N2CCc3ccccc3C2)cc1] | Method A | 105 | 10 uM |
| 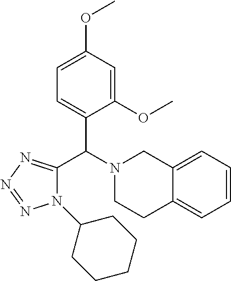 [SMILES: COc1ccc(C(c2nnnn2C2CCCCC2)N2CCc3ccccc3C2)c(OC)c1] | Method A | 113 | 10 uM |
| 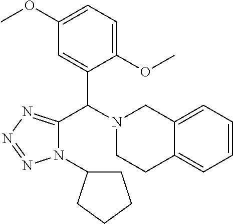 [SMILES: COc1ccc(OC)c(C(c2nnnn2C2CCCC2)N2CCc3ccccc3C2)c1] | Method A | 92 | 10 uM |
| 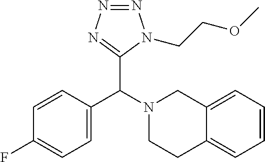 [SMILES: COCCn1nnnc1C(c1ccc(F)cc1)N1CCc2ccccc2C1] | Method A | 96 | 10 uM |
| 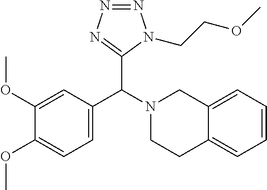 [SMILES: COCCn1nnnc1C(c1ccc(OC)c(OC)c1)N1CCc2ccccc2C1] | Method A | 98 | 10 uM |
| 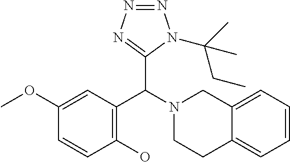 [SMILES: CCC(C)(C)n1nnnc1C(c1cc(OC)ccc1O)N1CCc2ccccc2C1] | Method A | 100 | 10 uM |
| 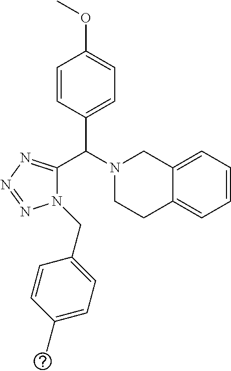 [SMILES: COc1ccc(C(c2nnnn2Cc2ccc(C)cc2)N2CCc3ccccc3C2)cc1] | Method A | 112 | 10 uM |
| 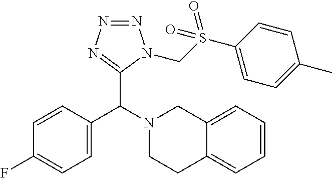 [SMILES: Cc1ccc(S(=O)(=O)Cn2nnnc2C(c2ccc(F)cc2)N2CCc3ccccc3C2)cc1] | Method A | 93 | 10 uM |
| 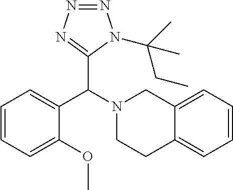 [SMILES: CCC(C)(C)n1nnnc1C(c1ccccc1OC)N1CCc2ccccc2C1] | Method A | 215 | 10 uM |
| 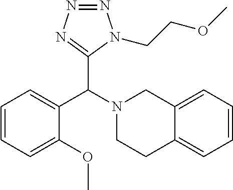 [SMILES: COCCn1nnnc1C(c1ccccc1OC)N1CCc2ccccc2C1] | Method A | 107 | 10 uM |
| 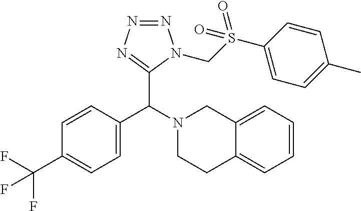 [SMILES: Cc1ccc(S(=O)(=O)Cn2nnnc2C(c2ccc(C(F)(F)F)cc2)N2CCc3ccccc3C2)cc1] | Method A | 102 | 10 uM |
| 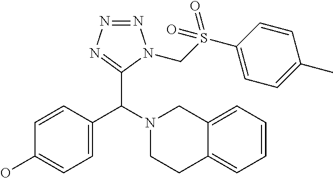 [SMILES: Cc1ccc(S(=O)(=O)Cn2nnnc2C(c2ccc(O)cc2)N2CCc3ccccc3C2)cc1] | Method A | 104 | 5 uM |

[symbol]

indicates data missing or illegible when filed

### Test Example 2

### Method B

Evaluation of Positive Allosteric Modulator Activity on PGI₂ Receptors

The positive allosteric modulator activity of a test compound was measured as a change in the amount of intracellular cyclic AMP (cAMP) in the presence of 1 nM of the IP agonist epoprostenol (manufactured by GlaxoSmithKline), using CHO-K1 cells (CHO/hIP cells) in which human PGI₂ receptors (hIP) are stably expressed.

An amount of 5 μL of a mixture (diluted in F-12 culture medium (manufactured by GIBCO) including 0.1% BSA) of epoprostenol dissolved in DMSO and a test compound dissolved in DMSO was dispensed in advance into a 384-well black microplate, and 5 μL of CHO/hIP cells suspended at a concentration of 1.4×10⁶ cells/mL in F-12 culture medium including 0.1% BSA were added to the microplate. After 40 minutes of incubation at room temperature, the amount of cAMP was measured using a cAMP assay kit (cAMP HiRange, manufactured by Cisbio Bioassays). Specifically, 5 μL of a cAMP-d2 solution (including a cell lysate) from the kit were added and the reaction was stopped, then 5 μL of a fluorescence-labeled cAMP antibody solution from the kit were added and incubation was performed for 1 hour at room temperature, after which fluorescence was measured using a PHERAstar plate reader (manufactured by BMG) in HTRF (registered trademark) mode. The amount of cAMP in each well was calculated from a cAMP calibration curve obtained using a standard sample, and the activity of each test compound is noted below as a ratio T/C (%) based on the amount of cAMP for 1 nM of epoprostenol alone. Results are shown in Table 4.

### Test Example 3

### Method C

Evaluation of Positive Allosteric Modulator Activity on PGI₂ Receptors

The effect of a test compound on the cAMP production curve with respect to the IP agonist epoprostenol (manufactured by GlaxoSmithKline) was investigated using CHO-K1 cells (CHO/hIP cells) in which human PGI₂ receptors (hIP) are stably expressed.

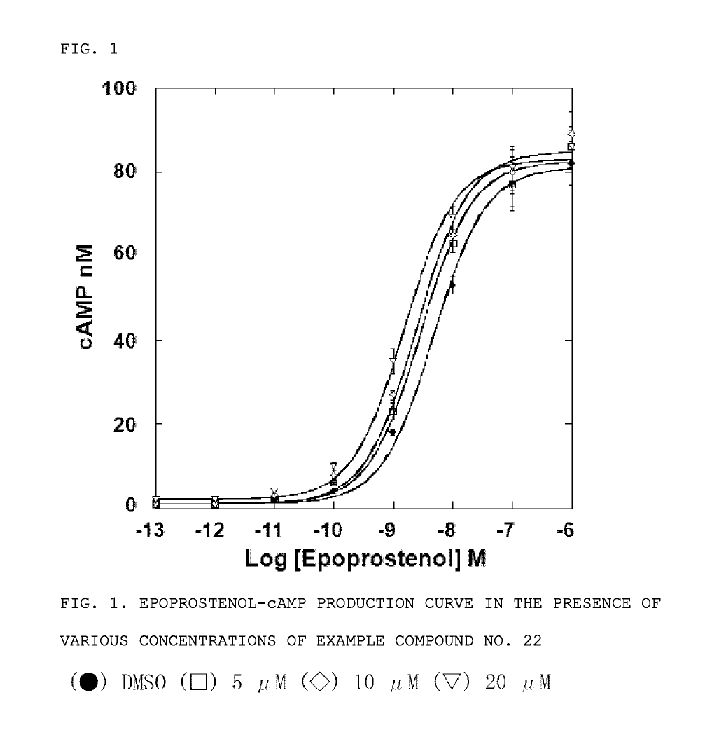

An amount of 5 μL of a mixture (diluted in F-12 culture medium (manufactured by GIBCO) including 0.1% BSA) of epoprostenol dissolved in DMSO and a test compound dissolved in DMSO was dispensed in advance into a 384-well black microplate, and 5 μL of CHO/hIP cells suspended at a concentration of 1.4×10⁶ cells/mL in F-12 culture medium including 0.1% BSA were added to the microplate. After 40 minutes of incubation at room temperature, the amount of cAMP was measured using a cAMP assay kit (cAMP HiRange, manufactured by Cisbio Bioassays). Specifically, 5 μL of a cAMP-d2 solution (including a cell lysate) from the kit were added and the reaction was stopped, then 5 μL of a fluorescence-labeled cAMP antibody solution from the kit were added and incubation was performed for 1 hour at room temperature, after which fluorescence was measured using a PHERAstar plate reader (manufactured by BMG) in HTRF (registered trademark) mode. The amount of cAMP in each well was calculated from a cAMP calibration curve obtained using a standard sample, and an epoprostenol-cAMP production curve for the presence of various concentrations of the test compound was created. From this curve, the EC₅₀ value of epoprostenol (the concentration of epoprostenol for producing 50% of the maximum amount of cAMP produced) was compared with the case of using epoprostenol alone to find the concentration of the test compound for producing a half concentration, and this concentration was used as the “left shift value.” FIG. 1 shows the epoprostenol-cAMP production curve for the presence of Example Compound No. 22. Results are shown in Tables 4 and 5. (In the tables, “ND” indicates that the left shift value could not be calculated.)

TABLE 4

| | Test | T/C [symbol] and left | Test |
|---|---|---|---|
| Example compound No. | method | shift value (uM) | concentration |
| 1 | Method B | 151.7 | 10 uM |
| | Method C | 24.9 | |
| 2 | Method B | 152.5 | 10 uM |
| | Method C | 15.5 | |
| 3 | Method B | 100.6 | 5 uM |
| | Method C | 71.4 | |
| 4 | Method B | 100.1 | 5 uM |
| | Method C | 35.7 | |
| 5 | Method B | 156.4 | 10 uM |
| | Method C | 10.9 | |
| 6 | Method B | 93.7 | 10 uM |
| | Method C | ND | |
| 7 | Method B | 127.6 | 10 uM |
| | Method C | 24.1 | |
| 8 | Method B | 184.2 | 10 uM |
| | Method C | 7.2 | |
| 9 | Method B | 153.2 | 10 uM |
| | Method C | 13.4 | |
| 10 | Method B | 117.0 | 10 uM |
| | Method C | 110.9 | |
| 11 | Method B | 95 | 10 uM |
| | Method C | ND | |
| 12 | Method B | 155.2 | 10 uM |
| | Method C | 10.4 | |
| 13 | Method B | 218.3 | 10 uM |
| | Method C | 3.1 | |
| 14 | Method B | 132.1 | 10 uM |
| | Method C | 17.7 | |
| 15 | Method B | 122.4 | 10 uM |
| | Method C | 32.4 | |
| 16 | Method B | 104.6 | 10 uM |
| | Method C | 232.2 | |
| 17 | Method B | 87 | 5 uM |
| | Method C | ND | |
| 18 | Method B | 94.1 | 10 uM |
| | Method C | ND | |
| 19 | Method B | 90 | 5 uM |
| | Method C | ND | |
| 20 | Method B | 126.3 | 5 uM |
| | Method C | 11.1 | |
| 21 | Method B | 229.7 | 10 uM |
| | Method C | 2.0 | |
| 22 | Method B | 149.6 | 10 uM |
| | Method C | 8.3 | |
| 23 | Method B | 106.4 | 10 uM |
| | Method C | 119.6 | |
| 24 | Method B | 116.3 | 10 uM |
| | Method C | 61.25 | |
| 25 | Method B | 177.2 | 10 uM |
| | Method C | 5.5 | |
| 26 | Method B | 105.5 | 10 uM |
| | Method C | 163.7 | |
| 27 | Method B | 99.8 | 5 uM |
| | Method C | 15.2 | |
| 28 | Method B | 99.1 | 5 uM |
| | Method C | 62.5 | |
| 29 | Method B | 135.5 | 10 uM |
| | Method C | 15.2 | |
| 30 | Method B | 100.4 | 10 uM |
| | Method C | 269.8 | |
| 31 | Method B | 186.2 | 10 uM |
| | Method C | 3.5 | |
| 32 | Method B | 153.8 | 10 uM |
| | Method C | 8.4 | |
| 33 | Method B | 119.7 | 10 uM |
| | Method C | 38.4 | |
| 34 | Method B | 145.9 | 10 uM |
| | Method C | 11.7 | |
| 35 | Method B | 121.8 | 10 uM |
| | Method C | 28.5 | |
| 36 | Method B | 172.6 | 10 uM |
| | Method C | 5.7 | |
| 37 | Method B | 104.9 | 10 uM |
| | Method C | 71.1 | |
| 38 | Method B | 147.0 | 10 uM |
| | Method C | 15.1 | |
| 39 | Method B | 144.4 | 10 uM |
| | Method C | 12.3 | |
| 40 | Method B | 89.3 | 10 uM |
| | Method C | ND | |
| 41 | Method B | 116.3 | 5 uM |
| | Method C | ND | |
| 42 | Method B | 142.5 | 10 uM |
| | Method C | 14.5 | |
| 43 | Method B | 267.8 | 10 uM |
| | Method C | 1.2 | |
| 44 | Method B | 143.8 | 5 uM |
| | Method C | 7.7 | |
| 45 | Method B | 144.3 | 5 uM |
| | Method C | 5.7 | |
| 46 | Method B | 106.2 | 10 uM |
| | Method C | 267.0 | |
| 47 | Method B | 121.9 | 10 uM |
| | Method C | 25.4 | |
| 48 | Method B | 72.3 | 10 uM |
| | Method C | ND | |
| 49 | Method B | 104.7 | 10 uM |
| | Method C | ND | |
| 50 | Method B | 105.0 | 10 uM |
| | Method C | ND | |
| 51 | Method C | 4.3 | |
| 52 | Method C | 4.3 | |
| 53 | Method C | 7.2 | |
| 54 | Method C | 4.7 | |
| 55 | Method C | 35 | |
| 56 | Method B | 101.0 | 10 uM |
| | Method C | 117.3 | |
| 57 | Method B | 256.5 | 10 uM |
| | Method C | 1.4 | |
| 58 | Method B | 102.7 | 10 uM |
| | Method C | 78.3 | |

[symbol]

indicates data missing or illegible when filed

TABLE 5

| Example | Test | | |
|---|---|---|---|
| compound No. | method | Left shift value (uM) | Test concentration |
| 59 | Method C | 16.1 | |
| 60 | Method C | 10.5 | |
| 61 | Method C | 1.5 | |
| 62 | Method C | 12.2 | |
| 63 | Method C | 34.9 | |
| 64 | Method C | 55.7 | |
| 65 | Method C | 66.2 | |
| 66 | Method C | 84.5 | |
| 67 | Method C | 32.4 | |
| 68 | Method C | 15.5 | |
| 69 | Method C | 20.6 | |
| 70 | Method C | 14.8 | |
| 71 | Method C | 2.3 | |
| 72 | Method C | 3.2 | |
| 73 | Method C | 44.6 | |
| 74 | Method C | 8.6 | |
| 75 | Method C | 2.1 | |
| 76 | Method C | 7.2 | |
| 77 | Method C | 16.3 | |
| 78 | Method C | 21.0 | |
| 79 | Method C | 12.3 | |
| 80 | Method C | 26.5 | |
| 81 | Method C | 8.3 | |
| 82 | Method C | 6.2 | |
| 83 | Method C | 25.0 | |

### Test Example 4

### Method D

Effect on cAMP Production in Human Aortic Smooth Muscle Cells

The effect of a test compound on the cAMP production curve with respect to the IP agonist epoprostenol (manufactured by GlaxoSmithKline) was investigated using human aortic smooth muscle cells known to express human PGI₂ receptors (hIP).

An amount of 5 μL of a mixture (diluted in F-12 culture medium (manufactured by GIBCO) including 0.1% BSA) of epoprostenol dissolved in DMSO and a test compound dissolved in DMSO was dispensed in advance into a 384-well black microplate, and 5 μL of CHO/hIP cells suspended at a concentration of 1.4×10⁶ cells/mL in F-12 culture medium including 0.1% BSA were added to the microplate. After 40 minutes of incubation at room temperature, the amount of cAMP was measured using a cAMP assay kit (cAMP HiRange, manufactured by Cisbio Bioassays). Specifically, 5 μL of a cAMP-d2 solution (including a cell lysate) from the kit were added and the reaction was stopped, then 5 μL of a fluorescence-labeled cAMP antibody solution from the kit were added and incubation was performed for 1 hour at room temperature, after which fluorescence was measured using a PHERAstar plate reader (manufactured by BMG) in HTRF (registered trademark) mode. The amount of cAMP in each well was calculated from a cAMP calibration curve obtained using a standard sample, and an epoprostenol-cAMP production curve for the presence of various concentrations of the test compound was created. From this curve, the EC₅₀ value of epoprostenol (the concentration of epoprostenol for producing 50% of the maximum amount of cAMP produced) was compared with the case of using epoprostenol alone to find the concentration of the test compound for producing a half concentration, and this concentration was used as the “left shift value.” Results are shown in Table 6.

TABLE 6

| Example compound No. | Test method | Left shift value (uM) |
|---|---|---|
| 51 | Method D | 3.9 |
| 54 | Method D | 2.9 |

### Test Example 5

### Magnus Test Using a Blood Vessel Removed from a Marmot

An incision was made in the thoracic region of a marmot (slc/Harley, male, 11-12 weeks of age) under anesthesia, and the thoracic aorta of the marmot was removed. The vascular endothelium was removed using a thread, after which the blood vessel was cut into a round slice having a width of approximately 2 mm and fixed to a fixing rod using thread or a stainless steel hook, and placed in a 10 mL Magnus tube maintained at a temperature of 37° C. and saturated with a 95% O₂/5% CO₂ gas mixture, and a tension of 1 g was placed on the blood vessel. When the tension on the specimen became stable, phenylephrine (phenylephrine hydrochloride, PHE, manufactured by Sigma-Aldrich Japan, final concentration: 3×10⁻⁶ mol/L) was added thereto to cause the specimen to contract, the test compound or a solvent was added when the contraction became stable, the IP agonist beraprost (beraprost sodium, manufactured by Cayman Chemical) was cumulatively added after 15 minutes to give a final concentration of 1 nM to 1000 nM, and the change in relaxation response was recorded.

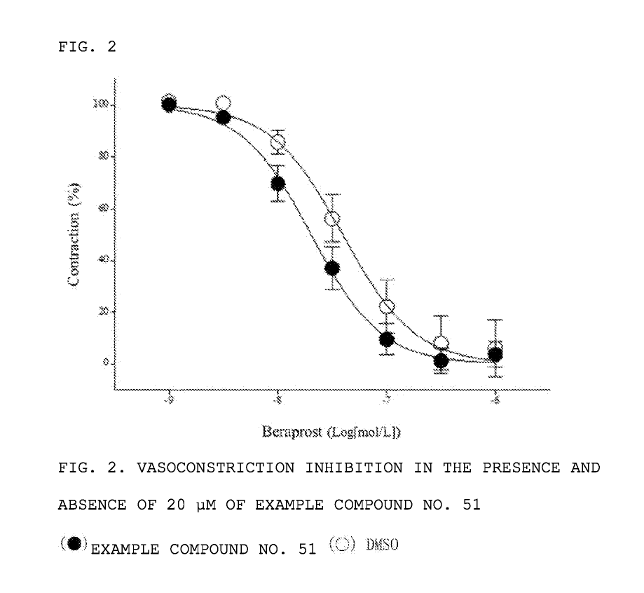

The contraction inhibition rate for each set of data was calculated using a contraction rate of 100% as the control (DMSO addition). The 50% vasoconstriction inhibition concentrations (ED₅₀ values) for beraprost were 18.3 μM and 32.1 μM for the presence and absence, respectively, of 20 μM of Example Compound No. 51. Results are shown in FIG. 2. Specifically, an enhancement of vasoconstriction inhibition (vasorelaxation) with respect to the IP agonist beraprost was recognized.

1. A positive allosteric regulator of a PGI₂ receptor which comprises a compound selected from Formulas (1) through (3) below or a pharmaceutically acceptable salt, hydrate, or solvate thereof 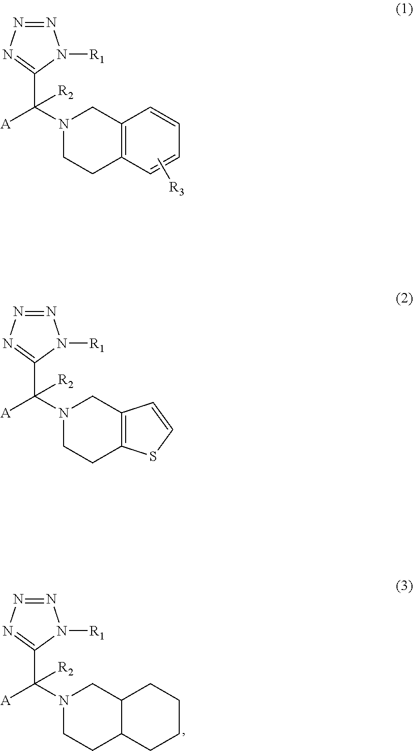
in which R¹ represents a branched-chain or a cyclic C₃₋₁₀ alkyl or alkenyl which may be substituted;
R² represents a hydrogen atom or a C₁₋₆ alkyl which may be substituted;
R³ represents 1 to 4 substituents, which are the same or different, independently selected from the group consisting of a hydrogen atom, a halogen atom, a C₁₋₆ alkyl which may be substituted, and a C₁₋₆ alkoxy which may be substituted; and
A represents an aryl which may be substituted or a heteroaryl which may be substituted.

2. The positive allosteric regulator according to claim 1, wherein R¹ is selected from tert-butyl, 1,1-dimethylpropyl, 1-methylcyclopentyl, 1-methylcyclohexyl, 1-methylcycloheptyl, 3-methyl-3-pentyl, and adamantyl.

3. The positive allosteric regulator according to claim 2, wherein R¹ is tert-butyl, 1,1-dimethylpropyl, or 1-methylcyclohexyl.

4. The positive allosteric regulator according to claim 1, wherein R² and R³ are both hydrogen atoms.

5. The positive allosteric regulator according to claim 1, wherein A is selected from the group consisting of phenyl, thienyl, furyl, pyrrolyl, indolyl, benzothiophenyl, and benzofuranyl, each of which may be substituted.

6. The positive allosteric regulator according to claim 1, wherein A is a group having the structure indicated below: 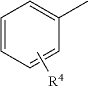
wherein R⁴ represents one to five substituents, which are the same or different, independently selected from the group consisting of a hydrogen atom, a C₁₋₆ alkyl which may be substituted, a C₁₋₆ alkoxy which may be substituted, a halogen atom, a hydroxyl group, a C₁₋₆ alkylthio which may be substituted, an amino which may be substituted, an acetylamino which may be substituted, a silylalkynyl which may be substituted, a benzyloxy which may be substituted, and a nitro; or, when two R⁴ groups are present, the two R⁴ groups may form a saturated or unsaturated ring structure which may include a hetero atom, together with carbon atoms to which the R⁴ groups are bonded.

7. The positive allosteric regulator according to claim 6, wherein R⁴ is selected from the group consisting of a hydrogen atom, a hydroxy group, an alkoxy group, a benzyloxy group, and an alkylthio group.

8. The positive allosteric regulator according to claim 7, wherein at least one R⁴ is a hydrogen atom, and at least one other R⁴ is a 2-hydroxy group, a 2-alkoxy group, a 2-benzyloxy group, or a 2-alkylthio group.

9. A tetrazole derivative selected from the group of compounds below or a pharmaceutically acceptable salt, hydrate, or solvate thereof:

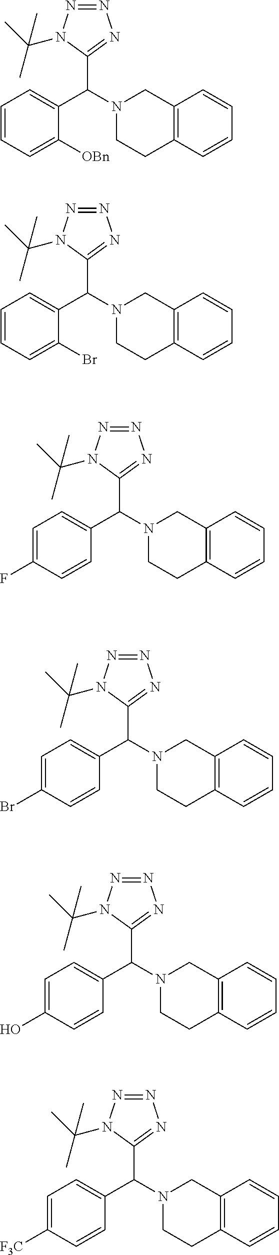 [SMILES: CC(C)(C)n1nnnc1C(c1ccccc1OCc1ccccc1)N1CCc2ccccc2C1 ; CC(C)(C)n1nnnc1C(c1ccccc1Br)N1CCc2ccccc2C1 ; CC(C)(C)n1nnnc1C(c1ccc(F)cc1)N1CCc2ccccc2C1 ; CC(C)(C)n1nnnc1C(c1ccc(Br)cc1)N1CCc2ccccc2C1 ; CC(C)(C)n1nnnc1C(c1ccc(O)cc1)N1CCc2ccccc2C1 ; CC(C)(C)n1nnnc1C(c1ccc(C(F)(F)F)cc1)N1CCc2ccccc2C1]

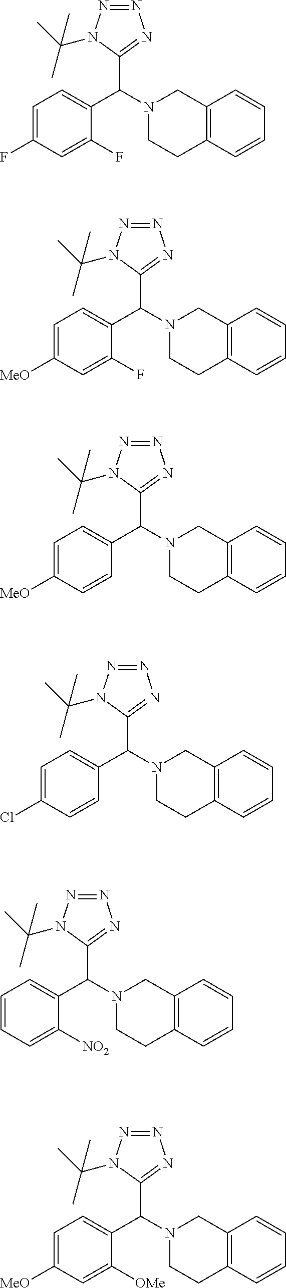 [SMILES: CC(C)(C)n1nnnc1C(c1ccc(F)cc1F)N1CCc2ccccc2C1 ; COc1ccc(C(c2nnnn2C(C)(C)C)N2CCc3ccccc3C2)c(F)c1 ; COc1ccc(C(c2nnnn2C(C)(C)C)N2CCc3ccccc3C2)cc1 ; CC(C)(C)n1nnnc1C(c1ccc(Cl)cc1)N1CCc2ccccc2C1 ; CC(C)(C)n1nnnc1C(c1ccccc1[N+](=O)[O-])N1CCc2ccccc2C1 ; COc1ccc(C(c2nnnn2C(C)(C)C)N2CCc3ccccc3C2)c(OC)c1]

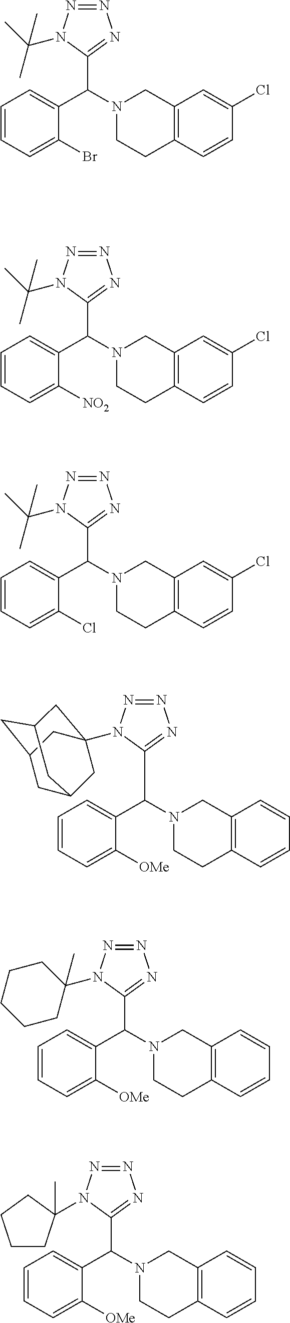 [SMILES: CC(C)(C)n1nnnc1C(c1ccccc1Br)N1CCc2ccc(Cl)cc2C1 ; CC(C)(C)n1nnnc1C(c1ccccc1[N+](=O)[O-])N1CCc2ccc(Cl)cc2C1 ; CC(C)(C)n1nnnc1C(c1ccccc1Cl)N1CCc2ccc(Cl)cc2C1 ; COc1ccccc1C(c1nnnn1C12CC3CC(CC(C3)C1)C2)N1CCc2ccccc2C1 ; COc1ccccc1C(c1nnnn1C1(C)CCCCC1)N1CCc2ccccc2C1 ; COc1ccccc1C(c1nnnn1C1(C)CCCC1)N1CCc2ccccc2C1]

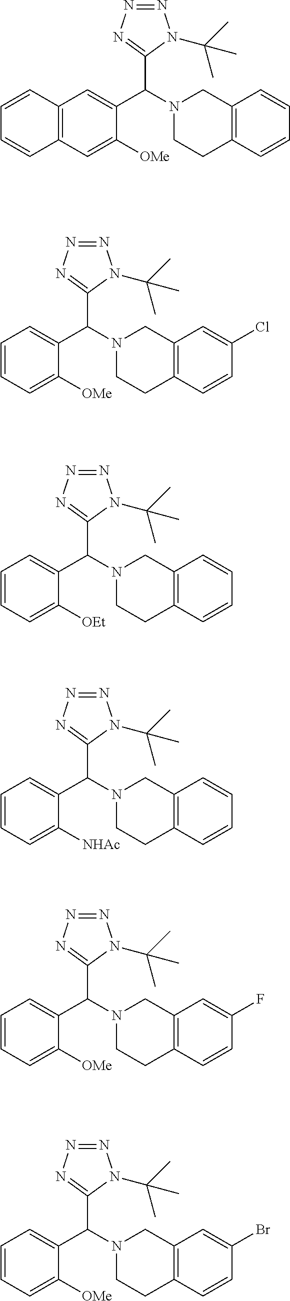 [SMILES: COc1cc2ccccc2cc1C(c1nnnn1C(C)(C)C)N1CCc2ccccc2C1 ; COc1ccccc1C(c1nnnn1C(C)(C)C)N1CCc2ccc(Cl)cc2C1 ; CCOc1ccccc1C(c1nnnn1C(C)(C)C)N1CCc2ccccc2C1 ; CC(=O)Nc1ccccc1C(c1nnnn1C(C)(C)C)N1CCc2ccccc2C1 ; COc1ccccc1C(c1nnnn1C(C)(C)C)N1CCc2ccc(F)cc2C1 ; COc1ccccc1C(c1nnnn1C(C)(C)C)N1CCc2ccc(Br)cc2C1]

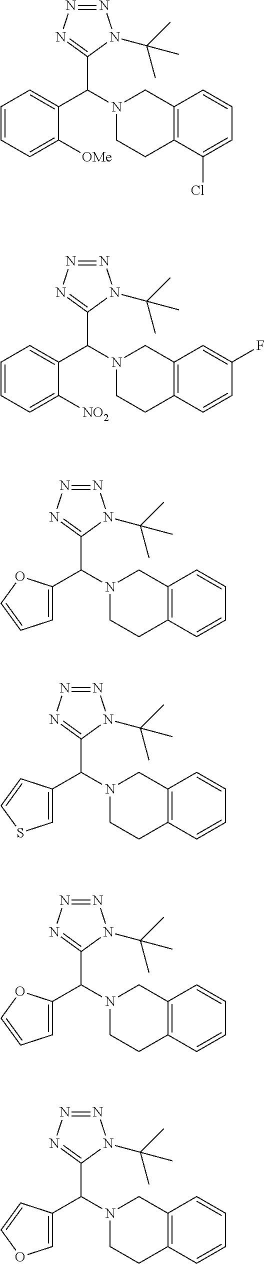 [SMILES: COc1ccccc1C(c1nnnn1C(C)(C)C)N1CCc2c(Cl)cccc2C1 ; CC(C)(C)n1nnnc1C(c1ccccc1[N+](=O)[O-])N1CCc2ccc(F)cc2C1 ; CC(C)(C)n1nnnc1C(c1ccco1)N1CCc2ccccc2C1 ; CC(C)(C)n1nnnc1C(c1ccsc1)N1CCc2ccccc2C1 ; CC(C)(C)n1nnnc1C(c1ccco1)N1CCc2ccccc2C1 ; CC(C)(C)n1nnnc1C(c1ccoc1)N1CCc2ccccc2C1]

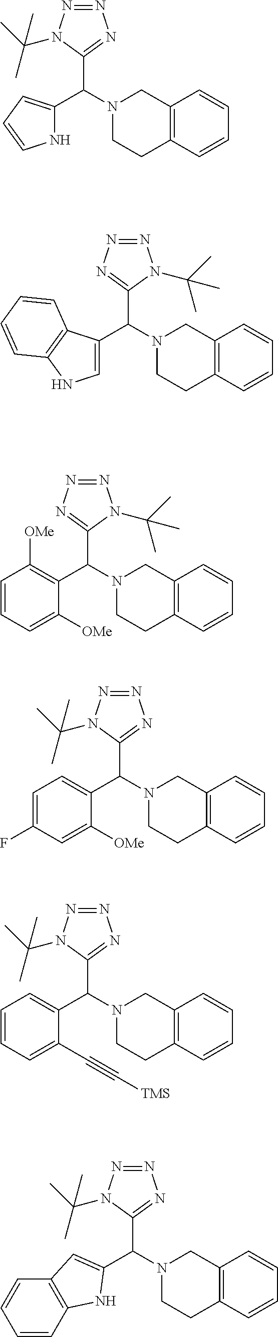 [SMILES: CC(C)(C)n1nnnc1C(c1ccc[nH]1)N1CCc2ccccc2C1 ; CC(C)(C)n1nnnc1C(c1c[nH]c2ccccc12)N1CCc2ccccc2C1 ; COc1cccc(OC)c1C(c1nnnn1C(C)(C)C)N1CCc2ccccc2C1 ; COc1cc(F)ccc1C(c1nnnn1C(C)(C)C)N1CCc2ccccc2C1 ; CC(C)(C)n1nnnc1C(c1ccccc1C#C[Si](C)(C)C)N1CCc2ccccc2C1 ; CC(C)(C)n1nnnc1C(c1cc2ccccc2[nH]1)N1CCc2ccccc2C1]

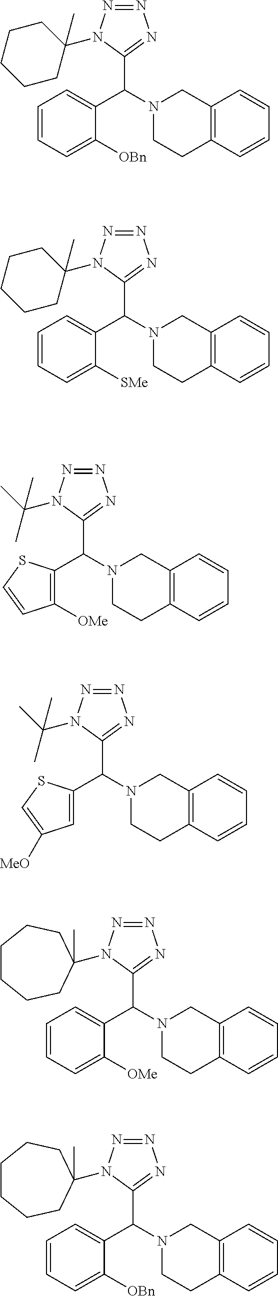 [SMILES: CC1(n2nnnc2C(c2ccccc2OCc2ccccc2)N2CCc3ccccc3C2)CCCCC1 ; CSc1ccccc1C(c1nnnn1C1(C)CCCCC1)N1CCc2ccccc2C1 ; COc1ccsc1C(c1nnnn1C(C)(C)C)N1CCc2ccccc2C1 ; COc1csc(C(c2nnnn2C(C)(C)C)N2CCc3ccccc3C2)c1 ; COc1ccccc1C(c1nnnn1C1(C)CCCCCC1)N1CCc2ccccc2C1 ; CC1(n2nnnc2C(c2ccccc2OCc2ccccc2)N2CCc3ccccc3C2)CCCCCC1]

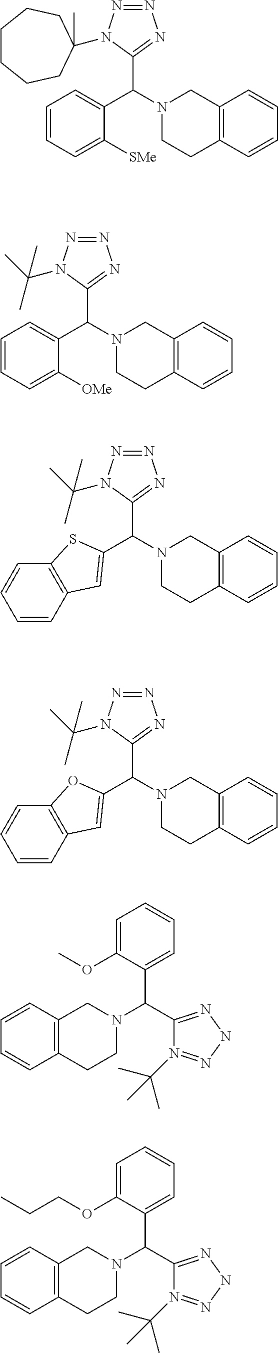

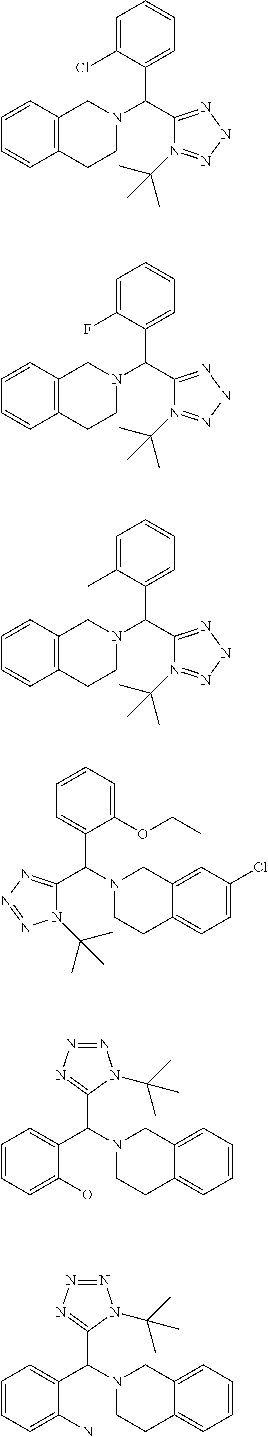

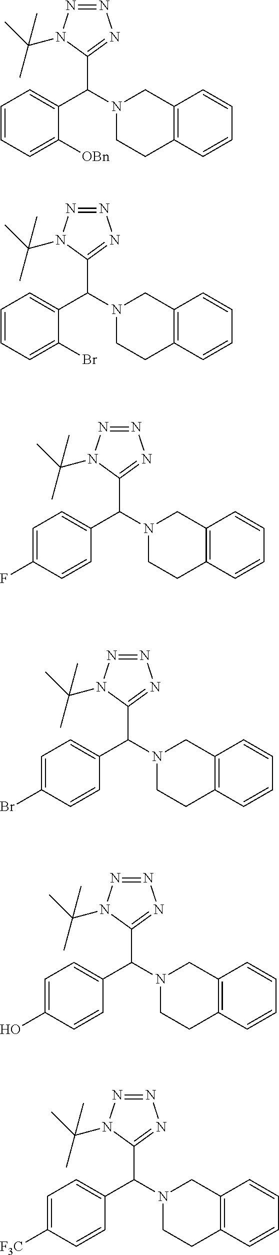 [SMILES: CC(C)(C)n1nnnc1C(c1ccccc1OCc1ccccc1)N1CCc2ccccc2C1 ; CC(C)(C)n1nnnc1C(c1ccccc1Br)N1CCc2ccccc2C1 ; CC(C)(C)n1nnnc1C(c1ccc(F)cc1)N1CCc2ccccc2C1 ; CC(C)(C)n1nnnc1C(c1ccc(Br)cc1)N1CCc2ccccc2C1 ; CC(C)(C)n1nnnc1C(c1ccc(O)cc1)N1CCc2ccccc2C1 ; CC(C)(C)n1nnnc1C(c1ccc(C(F)(F)F)cc1)N1CCc2ccccc2C1]

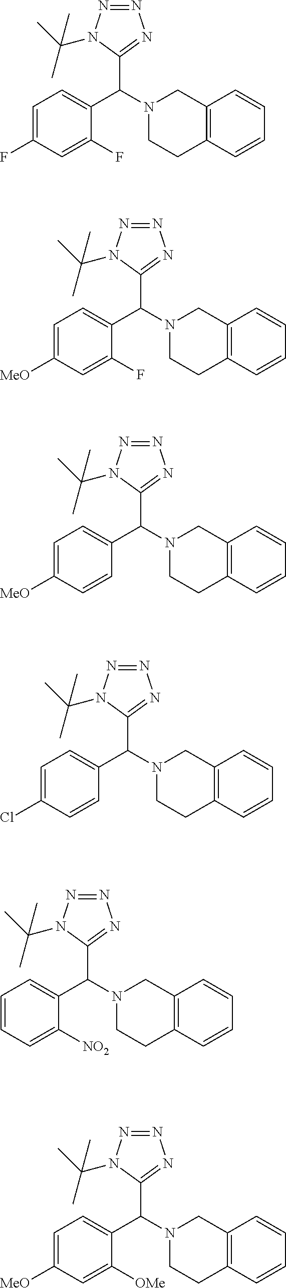 [SMILES: CC(C)(C)n1nnnc1C(c1ccc(F)cc1F)N1CCc2ccccc2C1 ; COc1ccc(C(c2nnnn2C(C)(C)C)N2CCc3ccccc3C2)c(F)c1 ; COc1ccc(C(c2nnnn2C(C)(C)C)N2CCc3ccccc3C2)cc1 ; CC(C)(C)n1nnnc1C(c1ccc(Cl)cc1)N1CCc2ccccc2C1 ; CC(C)(C)n1nnnc1C(c1ccccc1[N+](=O)[O-])N1CCc2ccccc2C1 ; COc1ccc(C(c2nnnn2C(C)(C)C)N2CCc3ccccc3C2)c(OC)c1]

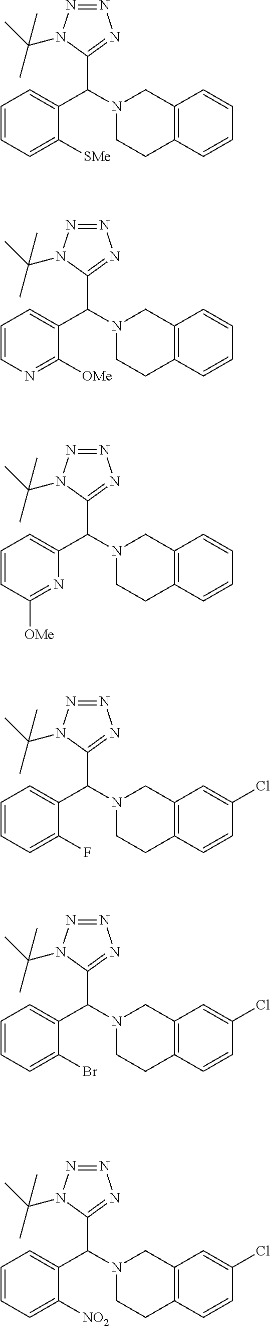 [SMILES: CSc1ccccc1C(c1nnnn1C(C)(C)C)N1CCc2ccccc2C1 ; COc1ncccc1C(c1nnnn1C(C)(C)C)N1CCc2ccccc2C1 ; COc1cccc(C(c2nnnn2C(C)(C)C)N2CCc3ccccc3C2)n1 ; CC(C)(C)n1nnnc1C(c1ccccc1F)N1CCc2ccc(Cl)cc2C1 ; CC(C)(C)n1nnnc1C(c1ccccc1Br)N1CCc2ccc(Cl)cc2C1 ; CC(C)(C)n1nnnc1C(c1ccccc1[N+](=O)[O-])N1CCc2ccc(Cl)cc2C1]

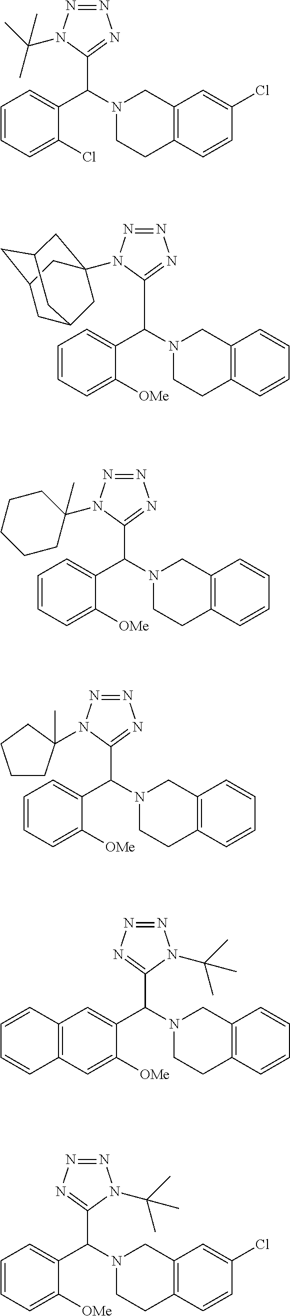 [SMILES: CC(C)(C)n1nnnc1C(c1ccccc1Cl)N1CCc2ccc(Cl)cc2C1 ; COc1ccccc1C(c1nnnn1C12CC3CC(CC(C3)C1)C2)N1CCc2ccccc2C1 ; COc1ccccc1C(c1nnnn1C1(C)CCCCC1)N1CCc2ccccc2C1 ; COc1ccccc1C(c1nnnn1C1(C)CCCC1)N1CCc2ccccc2C1 ; COc1cc2ccccc2cc1C(c1nnnn1C(C)(C)C)N1CCc2ccccc2C1 ; COc1ccccc1C(c1nnnn1C(C)(C)C)N1CCc2ccc(Cl)cc2C1]

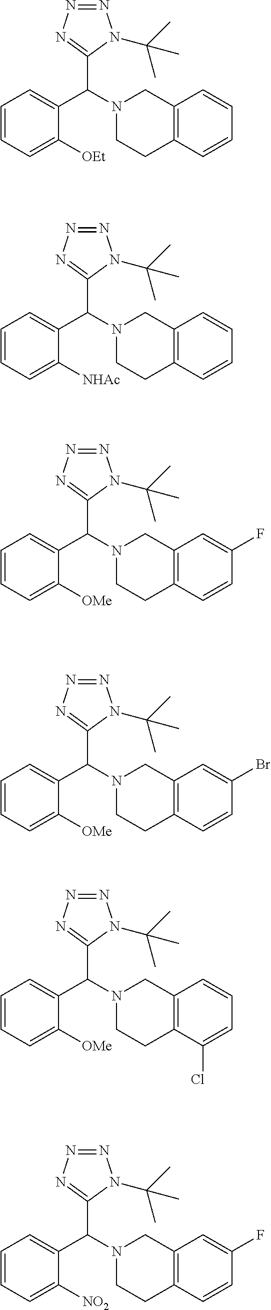 [SMILES: CCOc1ccccc1C(c1nnnn1C(C)(C)C)N1CCc2ccccc2C1 ; CC(=O)Nc1ccccc1C(c1nnnn1C(C)(C)C)N1CCc2ccccc2C1 ; COc1ccccc1C(c1nnnn1C(C)(C)C)N1CCc2ccc(F)cc2C1 ; COc1ccccc1C(c1nnnn1C(C)(C)C)N1CCc2ccc(Br)cc2C1 ; COc1ccccc1C(c1nnnn1C(C)(C)C)N1CCc2c(Cl)cccc2C1 ; CC(C)(C)n1nnnc1C(c1ccccc1[N+](=O)[O-])N1CCc2ccc(F)cc2C1]

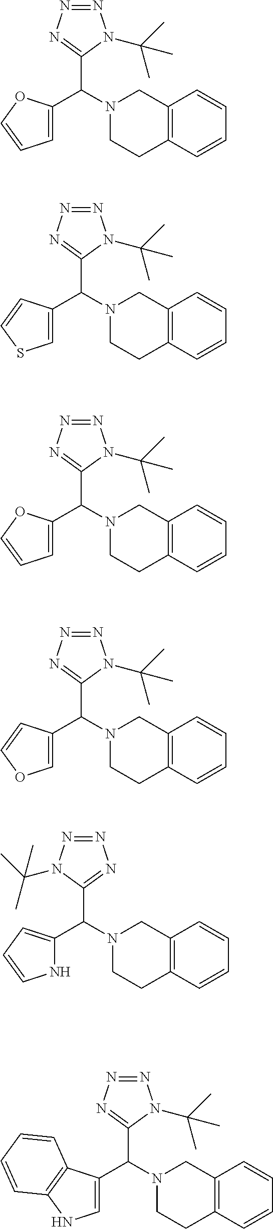 [SMILES: CC(C)(C)n1nnnc1C(c1ccco1)N1CCc2ccccc2C1 ; CC(C)(C)n1nnnc1C(c1ccsc1)N1CCc2ccccc2C1 ; CC(C)(C)n1nnnc1C(c1ccco1)N1CCc2ccccc2C1 ; CC(C)(C)n1nnnc1C(c1ccoc1)N1CCc2ccccc2C1 ; CC(C)(C)n1nnnc1C(c1ccc[nH]1)N1CCc2ccccc2C1 ; CC(C)(C)n1nnnc1C(c1c[nH]c2ccccc12)N1CCc2ccccc2C1]

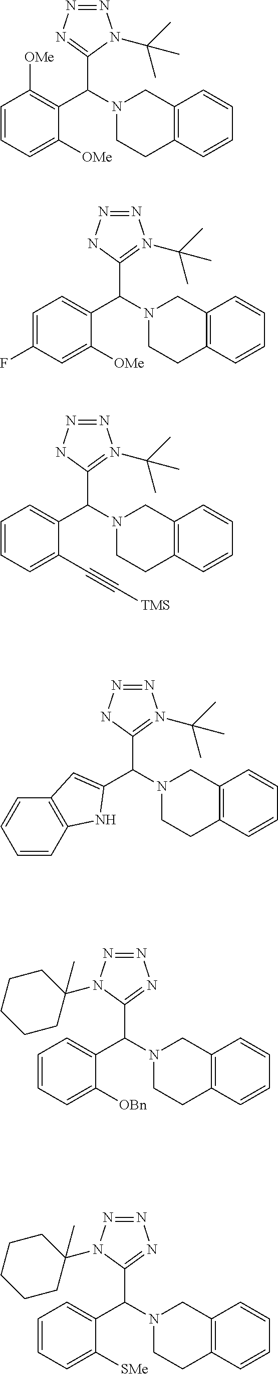

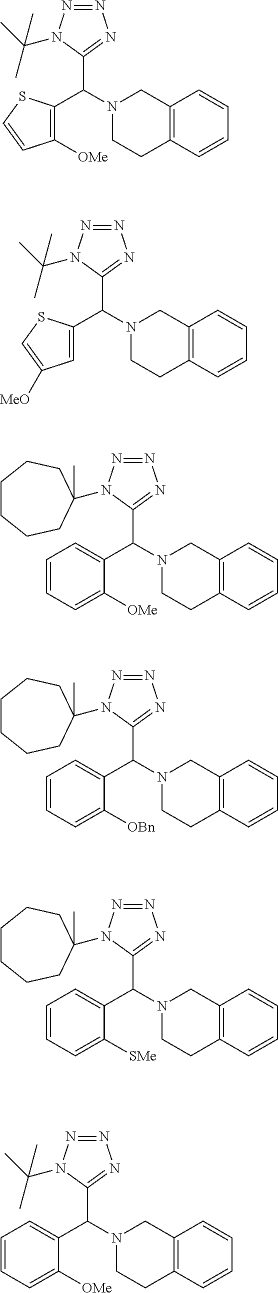 [SMILES: COc1ccsc1C(c1nnnn1C(C)(C)C)N1CCc2ccccc2C1 ; COc1csc(C(c2nnnn2C(C)(C)C)N2CCc3ccccc3C2)c1 ; COc1ccccc1C(c1nnnn1C1(C)CCCCCC1)N1CCc2ccccc2C1 ; CC1(n2nnnc2C(c2ccccc2OCc2ccccc2)N2CCc3ccccc3C2)CCCCCC1 ; CSc1ccccc1C(c1nnnn1C1(C)CCCCCC1)N1CCc2ccccc2C1 ; COc1ccccc1C(c1nnnn1C(C)(C)C)N1CCc2ccccc2C1]

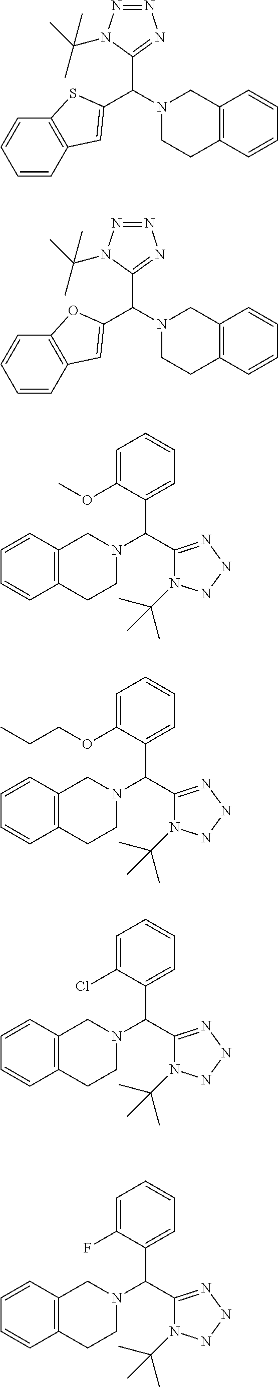

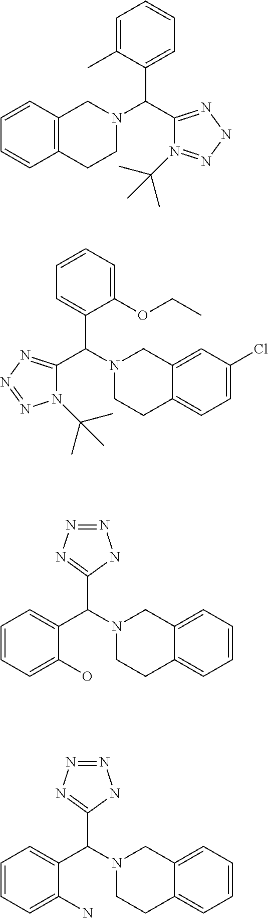

10. A compound represented by Formula (2) or (3) below or a pharmaceutically acceptable salt, hydrate, or solvate thereof: 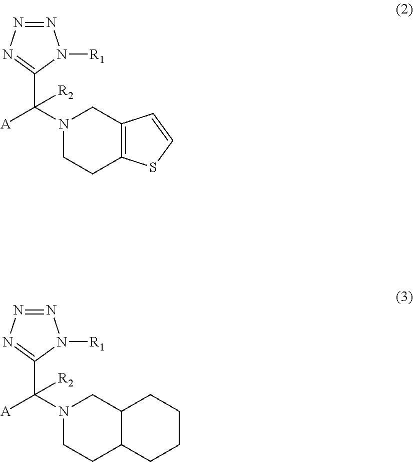
in which R¹ represents a branched-chain or cyclic C₃₋₁₀ alkyl or alkenyl which may be substituted;
R² represents a hydrogen atom or a C₁₋₆ alkyl which may be substituted; and
A represents an aryl which may be substituted or a heteroaryl which may be substituted.

11. A positive allosteric regulator of PGI₂ receptor comprising the compound according to claim 9, or pharmaceutically acceptable salt, hydrate, or solvate thereof.

12. A medical composition for treating or preventing disease induced by functional depression of PGI₂ receptors, wherein the medical composition comprises the positive allosteric regulator according to claim 1.

13. The medical composition according to claim 12, wherein said disease induced by functional depression of PGI₂ receptors is pulmonary hypertension.

14. A platelet aggregation inhibitor comprising the positive allosteric regulator according to claim 1.

15. A combination medicine for treating or preventing disease induced by functional depression of PGI₂ receptors, wherein the combination medicine comprises: (A) the positive allosteric regulator according to claim 1; and (B) a PGI₂ receptor agonist.

16. The combination medicine according to claim 15, wherein the PGI₂ receptor agonist is selected from the group consisting of epoprostenol, iloprost, cicaprost, beraprost, ibudilast, ozagrel, isbogrel, carbaprostacyclin, clinprost, ataprost, ciprostene, naxaprostene, taprostene, pimilprost, and phthalazinol.

17. The combination medicine according to claim 15, wherein said disease induced by functional depression of PGI₂ receptors is pulmonary hypertension.

18. A method of treating or preventing disease induced by functional depression of PGI₂ receptors comprising administering to a patient in need of such treatment a compound selected from Formulas (1) through (3) below: 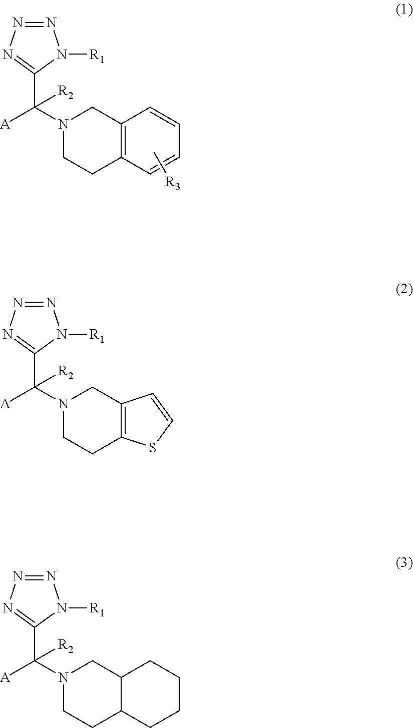
in which R¹ represents a branched-chain or cyclic C₃₋₁₀ alkyl or alkenyl which may be substituted;
R² represents a hydrogen atom or a C₁₋₆ alkyl which may be substituted;
R³ represents 1 to 4 substituents, which are the same or different, independently selected from the group consisting of a hydrogen atom, a halogen atom, a C₁₋₆ alkyl which may be substituted, and a C₁₋₆ alkoxy which may be substituted; and
A represents an aryl which may be substituted or a heteroaryl which may be substituted.
